SEQUENCE LISTING (45 sequences)

SEQ ID NO: 1 - protein, 70 aa, Homo sapiens
GPETLCGAELVDALQFVCGDRGFYFNKPTGYGSSSRRAPQTGIVDECCFRSCDLRRLEMYCAPLKPAKSA

SEQ ID NO: 2 - protein, 70 aa, synthetic construct
  REGION 1..70: note = synthetic construct
GPRTLCGAELVDALQFVCGDRGFYFNKPTGAGSSSRRAPQTGIVDECCFRSCDLRRLEMYCAPLKPAKSA

SEQ ID NO: 3 - protein, 83 aa, synthetic construct
  REGION 1..83: note = synthetic construct
MFPAMPLSSLFVNGPETLCGAELVDALQFVCGDRGFYFNKPTGYGSSSRRAPQTGIVDECCFRSCDLRRLEMYCAPLKPAKSA

SEQ ID NO: 4 - protein, 70 aa, synthetic construct
  REGION 1..70: note = synthetic construct
GPRTLCGAELVDALQFVCGDRGFYFNKPTGYGSSSRRAPQTGIVDECCFRSCDLRRLEMYCAPLKPAKSA

SEQ ID NO: 5 - protein, 67 aa, synthetic construct
  REGION 1..67: note = synthetic construct
TLCGAELVDALQFVCGDRGFYFNKPTGYGSSSRRAPQTGIVDECCFRSCDLRRLEMYCAPLKPAKSA

SEQ ID NO: 6 - protein, 83 aa, synthetic construct
  REGION 1..83: note = synthetic construct
MFPAMPLSSLFVNGPRTLCGAELVDALQFVCGDRGFYFNKPTGYGSSSRRAPQTGIVDECCFRSCDLRRLEMYCAPLKPAKSA

SEQ ID NO: 7 - protein, 69 aa, synthetic construct
  REGION 1..69: note = synthetic construct
GPETLCGAELVDALQFVCGDRGFYFNKPTGYGSSSRAPQTGIVDECCFRSCDLRRLEMYCAPLKPAKSA

SEQ ID NO: 8 - protein, 67 aa, synthetic construct
  REGION 1..67: note = synthetic construct
GPETLCGAELVDALQFVCGDRGFYFNKPTGYGSSSRRAPQTGIVDECCFRSCDLRRLEMYCAPLKPA

SEQ ID NO: 9 - protein, 319 aa, Homo sapiens
AQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 10 - protein, 319 aa, synthetic construct
  REGION 1..319: note = synthetic construct
AQVLRGTVTDFPGFDERADAETLRKAMKGLGTDEESILTLLTSRSNAQRQEISAAFKTLFGADLLDDLASELTGKFEKLIVALMKPSRLYDAYELKHALAGAGTNEKVLTEIIASRTPEELRAIKQVYEEEYGSSLAGDVVGDTSGYYQRMLVVLLQAARDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLAGEDD

SEQ ID NO: 11 - protein, 319 aa, synthetic construct
  REGION 1..319: note = synthetic construct
AQVLRGTVTDFPGFDERADAETLRKAMKGLGTDEESILTLLTSRSNAQRQEISAAFKTLFGADLLDDLASELTGKFEKLIVALMKPSRLYDAYELKHALAGAGTNEKVLTEIIASRTPEELRAIKQVYEEEYGSSLAGDVVGDTSGYYQRMLVVLLQAARDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 12 - protein, 585 aa, Homo sapiens [sequence omitted]

SEQ ID NO: 13 - protein, 585 aa, synthetic construct
  REGION 1..585: note = synthetic construct
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGL

SEQ ID NO: 14 - protein, 585 aa, synthetic construct
  REGION 1..585: note = synthetic construct
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAGTFTFHADICTLSEKERQIKKQTALVELVKHKPKATKEQLKAAMDDFAAFVEKCCKADDKETCFAEEGKKLVAASQAALGL

SEQ ID NO: 15 - protein, 585 aa, synthetic construct; identical to SEQ ID NO: 13
  REGION 1..585: note = synthetic construct

SEQ ID NO: 16 - protein, 20 aa, synthetic construct
  REGION 1..20: note = synthetic construct
GGGGSGGGGSGGGGSGGGGS

SEQ ID NO: 17 - protein, 698 aa, Homo sapiens
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAIAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 18 - protein, 609 aa, Homo sapiens
MKWVESIFLIFLLNFTESRTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 19 - protein, 474 aa, Homo sapiens
MKRVLVLLLAVAFGHALERGRDYEKNKVCKEFSHLGKEDFTSLSLVLYSRKFPSGTFEQVSQLVKEVVSLTEACCAEGADPDCYDTRTSALSAKSCESNSPFPVHPGTAECCTKEGLERKLCMAALKHQPQEFPTYVEPTNDEICEAFRKDPKEYANQFMWEYSTNYGQAPLSLLVSYTKSYLSMVGSCCTSASPTVCFLKERLQLKHLSLLTTLSNRVCSQYAAYGEKKSRLSNLIKLAQKVPTADLEDVLPLAEDITNILSKCCESASEDCMAKELPEHTVKLCDNLSTKNSKFEDCCQEKTAMDVFVCTYFMPAAQLPELPDVELPTNKDVCDPGNTKVMDKYTFELSRRTHLPEVFLSKVLEPTLKSLGECCDVEDSTTCFNAKGPLLKKELSSFIDKGQELCADYSENTFTEYKKKLAERLKAKLPDATPTELAKLVNKHSDFASNCCSINSPPLYCDSEIDAELKNIL

SEQ ID NO: 20 - protein, 147 aa, Homo sapiens
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 21 - protein, 20 aa, synthetic construct
  REGION 1..20: note = synthetic construct
ASPAAPAPASPAAPAPSAPA

SEQ ID NO: 22 - protein, 107 aa, synthetic construct
  REGION 1..107: note = synthetic construct
DIQMTQSPSSLSASVGDRVTITCRASQWIGSQLSWYQQKPGKAPKLLIMWRSSLQSGVPSRFSGSGSGTDFTLTISSLQPEDFATYYCAQGAALPRTFGQGTKVEIK

SEQ ID NO: 23 - protein, 7 aa, synthetic construct
  REGION 1..7: note = synthetic construct
GSGGGSG

SEQ ID NO: 24 - protein, 988 aa, synthetic construct
  REGION 1..988: note = synthetic construct
GPRTLCGAELVDALQFVCGDRGFYFNKPTGAGSSSRRAPQTGIVDECCFRSCDLRRLEMYCAPLKPAKSAGSGGGSGDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGLGSGGGSGAQVLRGTVTDFPGFDERADAETLRKAMKGLGTDEESILTLLTSRSNAQRQEISAAFKTLFGADLLDDLASELTGKFEKLIVALMKPSRLYDAYELKHALAGAGTNEKVLTEIIASRTPEELRAIKQVYEEEYGSSLAGDVVGDTSGYYQRMLVVLLQAARDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLAGEDD

SEQ ID NO: 25 - protein, 70 aa, synthetic construct
  REGION 1..70: note = Synthetic construct
GPRTLCGAELVDALQFVCGDRGFLFNKPTGYGSSSRRAPQTGIVDECCFRSCDLRRLEMYCAPLKPAKSA

SEQ ID NO: 26 - protein, 70 aa, synthetic construct
  REGION 1..70: note = Synthetic construct
GPRTLCGAELVDALQFVCGDRGFLFNKPTGYGSSSRRAPQTGIVDECCFRSCDLRRLEMLCAPLKPAKSA

SEQ ID NO: 27 - protein, 70 aa, synthetic construct
  REGION 1..70: note = Synthetic construct
GPRTLCGAELVDALQFVCGDRGFLFNKPTGAGSSSRRAPQTGIVDECCFRSCDLRRLEMYCAPLKPAKSA

SEQ ID NO: 28 - protein, 70 aa, synthetic construct; identical to SEQ ID NO: 26
  REGION 1..70: note = Synthetic construct

SEQ ID NO: 29 - protein, 70 aa, synthetic construct
  REGION 1..70: note = Synthetic construct
GPRTLCGAELVDALQFVCGDRGFYFNKPTGAGSSSRRAPQTGIVDECCFRSCDLRRLEMLCAPLKPAKSA

SEQ ID NO: 30 - protein, 70 aa, synthetic construct
  REGION 1..70: note = Synthetic construct
GPRTLCGAELVDALQFVCGDRGFLFNKPTGAGSSSRRAPQTGIVDECCFRSCDLRRLEMLCAPLKPAKSA

SEQ ID NO: 31 - protein, 72 aa, synthetic construct
  REGION 1..72: note = Synthetic construct
GTSHLVKCAEKEKTFCVNGGECFMVKDLSNPSRYLCKCPNEFTGDRCQNYVMASFYKHLGIEFMEAEELYQK

SEQ ID NO: 32 - protein, 72 aa, synthetic construct
  REGION 1..72: note = Synthetic construct
STSHLVKCAEKEKTFCVNGGECFMVKDLSNPSRYLCKCPNEFTGDRCQNYVMASFYKHLGIEFMEAEELYQK

SEQ ID NO: 33 - protein, 68 aa, synthetic construct
  REGION 1..68: note = Synthetic construct
TGTSHLVKCAEKEKTFCVNGGECFMVKDLSNPSRYLCKCQPGFTGARCTENVPMKVQNQEKAEELYQK

SEQ ID NO: 34 - protein, 76 aa, synthetic construct
  REGION 1..76: note = Synthetic construct
TGTSHLVKCAEKEKTFCVNGGECFMVKDLSNPSRYLCKCQPGFTGARCTENVPMKVQNQEKHLGIEFIEAEELYQK

SEQ ID NO: 35 - protein, 74 aa, synthetic construct
  REGION 1..74: note = Synthetic construct
TGTSHLVKCAEKEKTFCVNGGECFMVKDLSNPSRYLCKCQPGFTGARCTENVPMKVQNQESAQMSLLVIAAKTT

SEQ ID NO: 36 - protein, 65 aa, synthetic construct
  REGION 1..65: note = Synthetic construct
TGTSHLVKCAEKEKTFCVNGGECFMVKDLSNPSRYLCKCPNEFTGDRCQNYVMASFYKAEELYQK

SEQ ID NO: 37 - protein, 68 aa, synthetic construct
  REGION 1..68: note = Synthetic construct
TGTSHLVKCAEKEKTFCVNGGECFMVKDLSNPSRYLCKCPNEFTGDRCQNYVMASFYSTSTPFLSLPE

SEQ ID NO: 38 - protein, 70 aa, synthetic construct
GPKTLCGAELVDALQFVCGDRGFYFNKPTGYGSSSRRAPQTGIVDECCFRSCDLRRLEMYCAPLKPAKSA

SEQ ID NO: 39 - protein, 70 aa, synthetic construct
GPKTLCGAELVDALQFVCGDRGFLFNKPTGYGSSSRRAPQTGIVDECCFRSCDLRRLEMYCAPLKPAKSA

SEQ ID NO: 40 - protein, 70 aa, synthetic construct
GPKTLCGAELVDALQFVCGDRGFYFNKPTGAGSSSRRAPQTGIVDECCFRSCDLRRLEMYCAPLKPAKSA

SEQ ID NO: 41 - protein, 70 aa, synthetic construct
GPKTLCGAELVDALQFVCGDRGFYFNKPTGYGSSSRRAPQTGIVDECCFRSCDLRRLEMLCAPLKPAKSA

SEQ ID NO: 42 - protein, 70 aa, synthetic construct
GPKTLCGAELVDALQFVCGDRGFLFNKPTGYGSSSRRAPQTGIVDECCFRSCDLRRLEMLCAPLKPAKSA

SEQ ID NO: 43 - protein, 70 aa, synthetic construct
GPKTLCGAELVDALQFVCGDRGFLFNKPTGAGSSSRRAPQTGIVDECCFRSCDLRRLEMLCAPLKPAKSA

SEQ ID NO: 44 - protein, 70 aa, synthetic construct
GPKTLCGAELVDALQFVCGDRGFYFNKPTGAGSSSRRAPQTGIVDECCFRSCDLRRLEMLCAPLKPAKSA

SEQ ID NO: 45 - protein, 70 aa, unidentified
GPKTLCGAELVDALQFVCGDRGFLFNKPTGAGSSSRRAPQTGIVDECCFRSCDLRRLEMYCAPLKPAKSA

(19) United States
(12) Patent Application Publication
(10) Pub. No.: US 2025/0144175 A1
(43) Pub. Date: May 8, 2025

(54) CHIMERIC PROTEINS FOR TREATMENT OF ACUTE RADIATION SYNDROME

(71) Applicant: Silver Creek Pharmaceuticals, Inc., San Francisco, CA (US)

(72) Inventors: Lakhmir S. Chawla, San Diego, CA (US); Kristopher M. Kuchenbecker, Phoenix, AZ (US)

(73) Assignee: Silver Creek Pharmaceuticals, Inc., San Francisco, CA (US)

(21) Appl. No.: 18/836,833

(22) PCT Filed: Feb. 15, 2023
(86) PCT No.: PCT/US2023/062663

§ 371 Date: Aug. 8, 2024

Related U.S. Application Data
(60) Provisional application No. 63/310,483, filed on Feb. 15, 2022.
(60) Provisional application No. 63/327,445, filed on Apr. 5, 2022.

(51) Int. Cl. A61K 38/18; A61K 38/17; A61K 38/30; A61P 1/00; C07K 14/435; C07K 14/475; C07K 14/65

(52) U.S. Cl. CPC A61K 38/1883; A61K 38/17; A61K 38/30; A61P 1/00; C07K 14/435; C07K 14/4756; C07K 14/65; C07K 2319/01

(57) ABSTRACT

Aspects of the present disclosure relate generally to chimeric proteins and pharmaceutical compositions comprising such chimeric proteins, and methods for using such chimeric proteins for treating acute radiation syndrome in subjects in thereof.

### RELATED APPLICATIONS

This application claims priority to and the benefit of U.S. Provisional Application No. 63/310,483, filed Feb. 15, 2022, and of U.S. Provisional Application No. 63/327,445, filed Apr. 5, 2022, the contents of each of which are incorporated herein by reference in their entireties.

### REFERENCE TO SEQUENCE LISTING

This specification includes a sequence listing submitted herewith, which includes the file entitled 132463-010902.xlm having the following size: 59,287 bytes which was created Feb. 15, 2023, the contents of which are incorporated by reference herein.

### TECHNICAL FIELD

Aspects of the present disclosure relates generally to chimeric proteins and pharmaceutical compositions comprising such chimeric proteins, and methods for using such chimeric proteins for mitigating or treating Acute Radiation Syndrome in subjects in thereof.

### BACKGROUND

Acute radiation poisoning is a syndrome that encompasses a multitude of signs and symptoms that arise from exposure to high levels of ionizing radiation.

### SUMMARY

Methods of mitigating radiation toxicity, reducing radiation damage, and treating damage caused by exposure to radiation are provided. In some embodiments, methods of preventing radiation sickness are provided. In some embodiments, methods of mitigating radiation sickness are provided. In other embodiments, methods of treating radiation sickness are provided. In some embodiments, methods of preventing Acute Radiation Syndrome are provided. In some embodiments, methods of mitigating Acute Radiation Syndrome are provided. In other embodiments, methods of treating Acute Radiation Syndrome are provided.

Methods of effecting radioprotection are provided herein. In some embodiments, the method comprises administering a composition comprising an effective amount of a chimeric protein to provide radioprotection to a subject in need thereof, wherein the chimeric protein comprises a targeting domain comprising human annexin 5 or variant thereof, and an activator domain comprising insulin-like growth factor (IGF-1), neuregulin (NRG) or variant thereof, wherein the composition is administered to the subject between 3 hours to 7 days prior to radiation exposure.

Methods of mitigating radiation injury are provided herein. In some embodiments, the method comprises administering to a subject in need thereof a composition comprising an effective amount of a chimeric protein, wherein the chimeric protein comprises a targeting domain comprising human annexin 5 (AnxV) or variant thereof, and an activator domain comprising insulin-like growth factor (IGF-1), neuregulin (NRG) or variant thereof, wherein the composition is administered once a day or multiple times a day to the subject between 1 and 21 days after the radiation exposure. In some embodiments, the composition is administered to the subject between 1 and 24 hours after radiation exposure.

Methods of effecting radioprotection and mitigating injury from radiation are provided herein. In some embodiments, the method comprises administering to a subject in need thereof a composition comprising an effective amount of a chimeric protein, wherein the chimeric protein comprises a targeting domain comprising human annexin 5 (AnxV) or variant thereof, and an activator domain comprising insulin-like growth factor (IGF-1), neuregulin (NRG) or variant thereof, wherein the composition is initially administered to the subject between 3 hours to 7 days prior to radiation exposure, and wherein the composition is subsequently administered once a day or multiple times a day to the subject between 1 hour and 21 days after the radiation exposure.

Methods of treating acute radiation syndrome (ARS) are provided herein. In some embodiments, the method comprises administering to a subject in need thereof a composition comprising a therapeutically effective amount of a chimeric protein, wherein the chimeric protein comprises a targeting domain comprising human annexin 5 (AnxV) or variant thereof, and an activator domain comprising insulin-like growth factor (IGF-1), neuregulin (NRG) or variant thereof, wherein the composition is administered within 1 to 72 days after radiation exposure.

In some embodiments, the ARS is one or more of gastrointestinal (GI-ARS), hematopoietic (H-ARS), pulmonary (P-ARS), cutaneous (C-ARS), and cerebrovascular (B-ARS). In some embodiments, the subject is a human.

In some embodiments, the composition further comprises at least one physiologically acceptable carrier.

In some embodiments, the chimeric protein further comprising a peptide, wherein the peptide extends the half-life of the chimeric protein.

In some embodiments, the chimeric protein is targeted to a cell comprising phosphatidyl serine at its surface.

In some embodiments, the chimeric protein is targeted to an apoptotic cell.

In some embodiments, the targeting domain is a non-internalizing variant of annexin 5. In some embodiments, the non-internalizing variant of annexin 5 comprises one or more mutations, wherein the one or more mutations comprises a substitution at the position corresponding to C316 and optionally at one or more positions corresponding to R63, K70, K101, E138, D139, N160, and combinations thereof.

In some embodiments, the chimeric protein is substantially not internalized by the cell.

In some embodiments, the activator domain of chimeric protein is a variant of human insulin-like growth factor IGF-1 comprising one or more mutations, wherein the one or more mutations consist of a substitution at one or more positions corresponding to E3, Y24, Y31, Y60, and combinations thereof.

In some embodiments, the chimeric protein further comprises a half-life modulator comprising a variant of human serum albumin (HSA) comprising one or more mutations, wherein the one or more mutations consist of a substitution at one or more positions corresponding to C58 and N527, and combinations thereof.

In some embodiments, the effective amount is between about 0.1 mg/kg to about 100 mg/kg. In some embodiments, the effective amount is between about 12 mg/kg to 25 mg/kg.

In some embodiments, the effective amount is effective to mitigate or treat radiation injury or ARS in the subject. In some embodiments, the method increases the subject's chance of survival following exposure to radiation. In some embodiments, the dose of radiation received by the subject would be lethal to the subject in the absence of the administration of the composition. In some embodiments, the radiation comprises ionizing radiation.

In some embodiments, the ARS is one or more of gastrointestinal (GI-ARS), hematopoietic (H-ARS), pulmonary (P-ARS), cutaneous (C-ARS), and cerebrovascular (B-ARS).

In some embodiments, the subject is a human.

In some embodiments, the chimeric protein is IGF1(E3R/Y31A)_lk7_HSA26-609(C58S/N527Q)_lk7_AnxV2-320(R63A/K70A/K101A/E138A/D139G/N160A/C316A).

Provided herein is a composition for use in a method of effecting radioprotection, comprising administering the composition comprising an effective amount of a chimeric protein to a subject in need thereof, wherein the chimeric protein comprises a targeting domain comprising human annexin 5 or variant thereof, and an activator domain comprising insulin-like growth factor (IGF-1), neuregulin (NRG) or variant thereof, wherein the composition is administered to the subject between 3 hours to 7 days prior to radiation exposure. In some embodiments, the administration of the composition effects radioprotection in the subject in need thereof.

Provided herein is a composition for use in a method of mitigating radiation injury, comprising administering the composition comprising an effective amount of a chimeric protein to a subject in need thereof, wherein the chimeric protein comprises a targeting domain comprising human annexin 5 (AnxV) or variant thereof, and an activator domain comprising insulin-like growth factor (IGF-1), neuregulin (NRG) or variant thereof, wherein the composition is administered once or multiple times a day to the subject between 1 hour and 21 days after radiation exposure. In some embodiments, the administration of the composition mitigates radiation injury in the subject in need thereof.

Provided herein is a composition for use in a method of effecting radioprotection and mitigating injury from radiation, comprising administering the composition comprising an effective amount of a chimeric protein to a subject in need thereof, wherein the chimeric protein comprises a targeting domain comprising human annexin 5 (AnxV) or variant thereof, and an activator domain comprising insulin-like growth factor (IGF-1), neuregulin (NRG) or variant thereof, wherein the composition is initially administered to the subject between 3 hours to 7 days prior to radiation exposure, wherein the composition is subsequently administered once a day or multiple times a day to the subject between 1 hour and 21 days after the radiation exposure. In some embodiments, the administration of the composition effects radioprotection and mitigates injury from radiation in the subject in need thereof.

Provided herein is composition in a method for treating acute radiation syndrome (ARS), comprising administering the composition comprising a therapeutically effective amount of a chimeric protein to a subject in need thereof, wherein the chimeric protein comprises a targeting domain comprising human annexin 5 (AnxV) or variant thereof, and an activator domain comprising insulin-like growth factor (IGF-1), neuregulin (NRG) or variant thereof, wherein the composition is administered to the subject within 1 to 72 days after radiation exposure. In some embodiments, the administration of the composition results in the amelioration of one or more symptoms of ARS.

### DESCRIPTION OF THE SEQUENCE LISTING

SEQ ID NO: 1 is the amino acid sequence of wild-type human IGF-1 (mature form).

SEQ ID NO: 2 is the amino acid sequence of a variant of wild-type human IGF-1 variant comprising E3R and Y31 A substitutions.

SEQ ID NO: 3 is the amino acid sequence of a variant of human IGF-1 (IGF-1 LONG).

SEQ ID NO: 4 is the amino acid sequence of a variant of human IGF-1 (IGF1 E3R).

SEQ ID NO: 5 is the amino acid sequence of a variant of human IGF-1(IGF-1Des 1-3).

SEQ ID NO: 6 is the amino acid sequence of a variant of human IGF-1 (IGF-1 LR3).

SEQ ID NO: 7 is the amino acid sequence of a variant of human IGF-1 (IGF1 R37X).

SEQ ID NO: 8 is the amino acid sequence of a variant of human IGF-1 with deletion of residues 68-70 (IGF1 3X).

SEQ ID NO: 9 is the amino acid sequence of wild type human annexin A5 (AnxV).

SEQ ID NO: 10 is the amino acid sequence of a variant of wild-type human annexin 5 comprising the amino acids 2-320 of wild type annexin 5 and the R63A, K70A, K101A, E138A, D139G, N160A and C316A substitutions.

SEQ ID NO: 11 is the amino acid sequence of a non-internalizing variant of human annexin A5 (ni-AnxV).

SEQ ID NO: 12 is the amino acid sequence of wild type Human Serum Albumin (HSA).

SEQ ID NO: 13 is the amino acid sequence of Human Serum Albumin variant mHSA (C34S, N503Q substitutions).

SEQ ID NO: 14 is the amino acid sequence of Human Serum Albumin variant mHSA7 (C34S, N503Q, E505G and V547A substitutions).

SEQ ID NO: 15 is the amino acid sequence of a variant of human serum albumin comprising the amino acids 26-609 of wild type human serum albumin and the C58S and N527Q substitutions.

SEQ ID NO: 16 is the amino acid sequence of a peptide linker.

SEQ ID NO: 17 is the amino acid sequence of human transferrin (Tf).

SEQ ID NO: 18 is the amino acid sequence of Human Alpha Fetoprotein (AFP).

SEQ ID NO: 19 is the amino acid sequence of Human Vitamin D Binding Protein (VDBP).

SEQ ID NO: 20 is the amino acid sequence of Human Transthyretin (TTR).

SEQ ID NO: 21 is the amino acid sequence of a PASylation motif.

SEQ ID NO: 22 is the amino acid sequence of the albumin-binding domain human antibody (aldudAB).

SEQ ID NO: 23 is the amino acid sequence of a peptide linker lk7.

SEQ ID NO: 24 is the amino acid sequence of chimeric protein Scp776.

SEQ ID NO: 25 is the amino acid sequence of a variant of human IGF-1 (IGF-1 E3R, Y24L).

SEQ ID NO: 26 is the amino acid sequence of a variant of human IGF-1 (IGF-1 E3R, Y60L).

SEQ ID NO: 27 is the amino acid sequence of a variant of human IGF-1 (IGF-1 E3R, Y24L, Y31A).

SEQ ID NO: 28 is the amino acid sequence of a variant of human IGF-1 (IGF-1 E3R, Y24L, Y60L).

SEQ ID NO: 29 is the amino acid sequence of a variant of human IGF-1 (IGF-1 E3R, Y31A, Y60L).

SEQ ID NO: 30 is the amino acid sequence of a variant of human IGF-1 (IGF-1 E3R, Y24L, Y31A, Y60L).

SEQ ID NO: 31 is the amino acid sequence of wild type human neuregulin Beta1.

SEQ ID NO: 32 is the amino acid sequence of a variant of human neuregulin Beta1 (GIS substitution).

SEQ ID NO: 33 is the amino acid sequence of wild type human neuregulin Alpha.

SEQ ID NO: 34 is the amino acid sequence of wild type human neuregulin Alpha1A.

SEQ ID NO: 35 is the amino acid sequence of wild type human neuregulin Alpha3.

SEQ ID NO: 36 is the amino acid sequence of wild type human neuregulin Beta2.

SEQ ID NO: 37 is the amino acid sequence of wild type human Glian growth factor GGF2 (isoform of neuregulin 1Beta).

SEQ ID NO: 38 is the amino acid sequence of a variant of human IGF-1 (IGF-1 E3K).

SEQ ID NO: 39 is the amino acid sequence of a variant of human IGF-1 (IGF-1 E3K, Y24L).

SEQ ID NO: 40 is the amino acid sequence of a variant of human IGF-1 (IGF-1 E3K, Y31A).

SEQ ID NO: 41 is the amino acid sequence of a variant of human IGF-1 (IGF-1 E3K, Y60L).

SEQ ID NO: 42 is the amino acid sequence of a variant of human IGF-1(IGF-1 E3K, Y24L, Y60L).

SEQ ID NO: 43 is the amino acid sequence of a variant of human IGF-1(IGF-1 E3K, Y24L, Y31A, Y60L).

SEQ ID NO: 44 is the amino acid sequence of a variant of human IGF-1(IGF-1 E3K, Y31A, Y60L).

SEQ ID NO: 45 is the amino acid sequence of a variant of human IGF-1(IGF-1 E3K, Y24L, Y31A).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the hydrophobic core positions of IGF-1.

FIG. 2 is a graph showing the log-rank test comparing the survival distributions of two groups of mice that were subjected to a whole body irradiation dose, with partial shielding, of 1650 cGy, at a targeted dose rate of 53 cGy per minute.

FIG. 3 is a graph showing the change in body weight for the two groups of mice that were subjected to a whole body irradiation dose, with partial shielding, of 1650 cGy, at a targeted dose rate of 53 cGy per minute.

FIG. 4 is a table showing the frequency of clinical signs observed for the two groups of mice that were subjected to a whole body irradiation dose, with partial shielding, of 1650 cGy, at a targeted dose rate of 53 cGy per minute.

FIG. 5 is a table showing the clinical laboratory results for the two groups of mice that were subjected to a whole body irradiation dose, with partial shielding, of 1650 cGy, at a targeted dose rate of 53 cGy per minute.

FIG. 6 is a table showing the macroscopic observations of intestinal damage for the two groups of mice that were subjected to a whole body irradiation dose, with partial shielding, of 1650 cGy, at a targeted dose rate of 53 cGy per minute.

### DETAILED DESCRIPTION

It is to be understood that the terminology used herein is for the purpose of describing particular embodiments of the disclosure only and is not intended to be limiting.

Unless defined otherwise, all technical and scientific terms used herein have the same meaning as commonly understood by one having ordinary skill in the art to which the disclosure pertains.

All publications, patents and patent applications cited herein, whether supra or infra, are hereby incorporated by reference in their entirety.

### Definitions

As used in this specification and the appended claims, the singular forms “a, “an” and “the” include plural referents unless the content clearly dictates otherwise.

The term “peptide,” “polypeptide” and “protein” are used interchangeably to denote a sequence polymer of at least two amino acids covalently linked by an amide bond (also referred herein as peptide bond).

As used herein the term “target molecule” refers to any molecule that is associated with a tissue (e.g. “at risk”, diseased or damaged tissue). As used herein the term “target molecule” refers to any molecule that in a healthy or undamaged cell is not substantially presented on the surface of the cell, but in a diseased or damaged cell, the “target molecule” is either expressed or redistributed towards substantial presentation on the surface of the cell. A “target cell” is meant to be a cell to which a protein or targeting domain thereof can specifically bind.

“Binding” or “specific binding” are used interchangeably herein and indicates that a protein (or the targeting polypeptide domain thereof or the activator domain thereof) exhibits substantial affinity for a specific molecule (e.g., targeting domain exhibits substantial affinity for a target molecule, or an activator domain exhibits substantial affinity for a molecule associated with the surface of a cell such as a growth factor receptor) or a cell or tissue bearing the molecule and is said to occur when the protein (or the targeting polypeptide domain thereof or the activator domain thereof) has a substantial affinity for a specific molecule and is selective in that it does not exhibit significant cross-reactivity with other molecules.

“Identity,” as known in the art, is a relationship between two or more polypeptide or protein sequences, as determined by comparing the sequences. In the art, “identity” also refers to the degree of sequence relatedness between polypeptides or proteins, as determined by the match between strings of such sequences. “Identity” can be readily calculated by any bioinformational methods known in the art.

The term “parent polypeptide” refers to a wild-type polypeptide and the amino acid sequence or nucleotide sequence of the wild-type polypeptide is part of a publicly accessible protein database (e.g., EMBL Nucleotide Sequence Database, NCBI Entrez, ExPasy, Protein Data Bank and the like).

The term “mutant polypeptide” or “polypeptide variant” refers to a form of a polypeptide, wherein its amino acid sequence differs from the amino acid sequence of its corresponding wild-type (parent) form, naturally existing form or any other parent form. A mutant polypeptide can contain one or more mutations, e.g., substitution, insertion, deletion, addition etc . . . which result in the mutant polypeptide. Generally, variants are overall closely similar, and, in many regions, identical to the reference polypeptide. As used herein, “variant” refers to a polypeptide, differing in sequence from a native protein but retaining at least one functional and/or therapeutic property thereof as described elsewhere herein or otherwise known in the art.

The term “corresponding to a parent polypeptide” is used to describe a polypeptide of the disclosure, wherein the amino acid sequence of the polypeptide differs from the amino acid sequence of the corresponding parent polypeptide only by the presence of at least one amino acid variation. Typically, the amino acid sequences of the variant polypeptide and the parent polypeptide exhibit a high percentage of identity. In one example, “corresponding to a parent polypeptide” means that the amino acid sequence of the variant polypeptide has at least about 50% identity, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98% identity or at least about 99% identity to the amino acid sequence of the parent polypeptide. In another example, the nucleic acid sequence that encodes the variant polypeptide has at least about 50% identity, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98% identity or at least about 99% identity to the nucleic acid sequence encoding the parent polypeptide.

The term “substantial identity” or “substantial similarity,” as used herein, when referring to a nucleic acid or fragment thereof, indicates that when optimally aligned with appropriate nucleotide insertions or deletions with another nucleic acid (or its complementary strand), there is nucleotide sequence identity in at least about 95% to 99% of the sequence. The term “substantial identity” or “substantial similarity,” as used herein, when referring to a protein or fragment thereof, indicates that when optimally aligned there is an amino acid sequence identity in at least about 95% to 99% of the sequence.

The term “damaged cell” or “damaged tissue,” as used herein, means and includes biological cell or tissue that is damaged following ionizing radiation exposure. In some embodiments, the damaged cells comprise apoptotic cells. In some embodiments, the damaged cells comprise hematopoietic stem cells. In some embodiments, the damaged cells comprise intestinal epithelial cells. In some embodiments, the damaged cells comprise cerebrovascular cells. In some embodiments, the damaged cells comprise lung cells. In some embodiments, the damaged cells comprise kidney cells. In some embodiments, the damaged cells comprise epithelial cells. In some embodiments, the damaged cells comprise hematopoietic cells. In some embodiments, the damaged cells comprise stem cells. In some embodiments, the damaged cells comprise neurons. In some embodiments, the damaged cells comprise leukocytes. In some embodiments, the damaged cells comprise cutaneous cells. In some embodiments, the damaged cells comprise cardiomyocytes.

The term “therapeutically effective amount,” as used herein, means the amount of the protein or agent that will elicit the biological or medical response of a tissue, system, animal or human that is being sought by the researcher, veterinarian, medical doctor or other clinician.

The term “pharmaceutically acceptable,” as used herein, means the carrier, diluent or excipient must be compatible with the other ingredients of the formulation and not deleterious to the recipient thereof.

The term “targeting moiety”, “targeting domain,” “targeting polypeptide” or “targeting module” are used herein interchangeably and refer to molecules that selectively localize the chimeric protein in a particular tissue or region of the body. The localization can be mediated by specific recognition of molecular determinants, molecular size of the targeting domain, ionic interactions, hydrophobic interactions and the like. As used herein, the terms “therapeutic moiety,” “activator domain,” “activator polypeptide”, “signaling arm” and “effector module” are used herein interchangeably and refers to any agents useful for therapy and that are non-toxic, do not have a cytotoxic effect or are not detrimental to the cells. Such agents can include, but not limited to, growth factors.

### Acute Radiation Syndrome

Acute radiation poisoning is a syndrome that encompasses a multitude of signs and symptoms that arise from the exposure to high levels of ionizing radiation. Ionizing radiation include alpha, beta, gamma and x-rays. As used herein, the terms “ionizing radiation” and “radiation” are used interchangeably. The primary source for such ionizing radiation is from nuclear weapons, nuclear reactors, and facilities that utilize radiation for the treatment of human disease (e.g. cancer).

The side effects associated with acute radiation poisoning radiation can be one or more of acute-nature nausea, vomiting, abdominal pain, diarrhea, dizziness, headache, fever, cutaneous radiation syndrome, low blood cell count, infection due to low white blood cells, bleeding due to low platelets, anemia due to low red blood cells, weight loss or death.

While not comprehensive, there are five main acute radiation syndromes (ARS): gastrointestinal (GI-ARS), hematopoietic (H-ARS), pulmonary (P-ARS), cutaneous (C-ARS), and cerebrovascular (B-ARS). Each of these syndromes describe the form of injury seen when a sufficiently high dose of radiation exposure occurs to those regions of the body.

H-ARS, defined as the development of neutropenia, thrombocytopenia and anemia, appears at the lowest end of the dose range resulting in injury; it occurs due to the radiosensitivity of committed progenitor cells in these lineages. Doses of ˜2 gray (Gy) result in decreased lymphocyte counts and immune suppression, making victims susceptible to secondary infections. Exposure may result in bone marrow failure and lethal hemorrhage or infections. Without recovery or treatment, death may occur within 2-4 weeks; therefore, protection or reconstitution of the hematopoietic systems is a major concern in the development of medical counter-measures (MCMs).

The GI tract is considered to be particularly sensitive to radiation exposure. Intestinal radiation injury, consisting of enterocyte depletion, mucosal barrier breakdown and mucositis with secretory diarrhea, occurs as a consequence of many concurrent and sequential pathophysiological events. Other elements of the intestine also contribute to system dysfunction: the enteric muscularis, immune system, microvasculature and nervous system and the resident bacteria and fungi, although classical GI injury is attributed to death of clonogenic crypt epithelial stem cells. The pathological aspect of GI injury emphasizes protein modifications, changes in redox status, secondary effects due to inflammation and release of cytokines and the functional consequences of cell loss. Cellular compartments may contribute to and modulate organ dysfunction, but the key event in the pathophysiology of GI injury is enterocyte depletion, with vascular damage possibly contributing at higher radiation doses. The physiological approach emphasizes early vomiting and diarrhea as common GI-related symptoms of exposure which greatly exacerbate the problem of fluid and electrolyte loss, which may result in death. Even at radiation doses below the threshold for GI syndrome, mucosal barrier breakdown allows bacteria to translocate into circulation, which can cause sepsis and death in the setting of concomitant immune suppression. Long term tissue remodeling after radiation damage alters the structure, motility and absorption of the gut; fibrosis renders it more rigid and susceptible to adhesions, stenosis and perforation.

The lung is a very sensitive organ which, when exposed to radiation, may result in acute and chronic inflammation that can lead to fatal lung fibrosis. The majority of animal models display a higher tolerance to pulmonary radiation than humans. Lung injury manifests within a few months after irradiation; inflammatory pneumonitis develops within 2-4 months after irradiation and fibrosis is observed after 4-6 months. Histologically, pneumonitis is characterized by interstitial and airspace edema, inflammatory infiltrate of predominantly macrophages and loss of epithelial cells.

Accidental exposure of the human skin to ionizing radiation>3 Gy results in a distinct clinical representation (C-ARS), which is characterized by a transient and faint erythema after a few hours, followed by severe erythema, blistering and necrosis. Depending on severity of damage, the latter generally occurs 10-30 days after exposure, but, in severe cases, it may appear within 48 hours. For C-ARS, the therapeutic approaches should include topical and systemic anti-inflammatory measures at the earliest conceivable point and should be maintained throughout the acute and subacute stages, as this reduces the need for surgical intervention, once necrosis has occurred.

Cerebrovascular symptoms (B-ARS) only occur at whole body doses in excess of 10 Gy. Also known as neurovascular sub-syndrome, it results from localized changes in the CNS, including impaired capillary circulation, damage to the blood-brain barrier, interstitial edema, acute inflammation, petechial hemorrhages, inflammation of the meninges and hypertrophy of perivascular astrocytes. Signs and symptoms include persistent and severe nausea, vomiting, accompanied by headache, neurological deficits, disorientation, confusion, loss of balance and seizures. Physical examination may show papilledema, ataxia and reduced or absent deep tendon and corneal reflexes.

Because damage resulting from such extremely high radiation exposure has been deemed untreatable, the scientific community has focused its efforts on finding preventative and mitigating treatments for only hematopoietic, pulmonary and GI syndromes of ARS.

Radiation nephropathy (RN) is a kidney injury induced by for example ionizing radiation.

In ischemic injuries (e.g. to heart, brain, kidney, etc . . . ), the damage can be considered as a core mass of injured tissue. The injury context of a damaged core and healthy surrounding areas is conceptually more accessible in explanation of targeted delivery of pro-survival signals. The damage in ARS has been found to be diffuse throughout a given organ system. Phosphatidyl-serine driven diffusion through interstitial space can allow specific delivery of chimeric proteins targeting phosphatidyl-serine to an archipelago of damaged cells rather than the single island catalyzed in ischemic injury.

### Chimeric Proteins

Chimeric proteins provided herein are capable of specific binding to two or more different specific molecules. In some embodiments, the chimeric protein comprises a targeting domain having a binding specificity to a first specific target molecule, an activator domain having a binding specificity to a second target molecule, and a half-life modulator.

In some aspects, the activator domain has a binding specificity with a IGF-1 receptor. Insulin-like growth factor 1 receptor (IGF-1R) is a transmembrane receptor tyrosine kinase whose activation strongly promotes cell growth and survival. IGF-1R exerts its main actions through the activation of the mitogen-activated protein kinase and phosphoinositide 3-kinase pathways.

In addition to their traditional roles, IGF-1R activation has been associated with increased radio-resistance both in vitro and in vivo, although the molecular mechanisms behind this process are still unclear.

Acute radiation injury induces both cell death and apoptosis. In some embodiments, the chimeric protein targets cell that are injured/damaged by radiation or apoptotic cells. In some embodiments, the chimeric protein activates the IGF-1 receptor pro-survival signaling pathways to both protect and mitigate H-ARS, GI-ARS, and P-ARS. In some aspects, the activator domain has a binding specificity to a receptor that modulates/promotes tissue regeneration.

In some aspects, the activator domain has a binding specificity with a HER2/HER3. HER2/HER3 is a transmembrane receptor tyrosine kinase whose activation strongly promotes cell growth and survival. Activated ERBB exerts its pro-survival actions through the mitogen-activated protein kinase and phosphoinositide 3-kinase pathways. As used herein the term “ErbB”, or “ERBB” or “HER” refers to the expression product (RNA or protein) of the ERBB gene, which is also frequently called HER (from human epidermal growth factor receptor) or HER/neu.

In some aspects, the activator domain has a binding specificity with ERBB receptor tyrosine kinases (RTKs). In some embodiments, the activator domain is neuregulin (NRG), fragment thereof, of variant thereof having a binding specificity with ERBB receptor tyrosine kinases. The ERBB RTKs are transmembrane receptors with extracellular ligand binding domains and intracellular kinase domains. The extracellular domain of ERBB2 does not have known ligands, whereas the intracellular domain of ERBB3 does not have a functional kinase domain. NRG1 is a ligand for the extracellular domain of ERBB3 that promotes heterodimerization with the other members of this family of RTKs. These heterodimers couple the potent ligand binding of neuregulin to ERBB3 with strong activation of the intracellular kinase domains of ERBB2 and ERBB4. The downstream signaling of these stimulated receptors converge in activation of Protein Kinase B (Akt), promoting cell survival and growth. Across different cancers, NRG1 expression levels have been correlated to proliferation and self-renewal with associated ERBB2 activation. In breast cancer cell lines, inhibition of the ERBB RTKs greatly enhance the sensitivity of cells to ionizing radiation (Lee et al., Cancer Res., 74(1): 341-352, 2014). As ARS is primarily driven through apoptosis, neuregulin activation of the pro-survival Akt pathways provides an apoptotic-exit signal to those cells that have suffered DNA breaks as a result of radiation damage. Without being bound to the theory, this exit from programmed cell death mitigates and protects against the damage from the ionizing injury.

In some aspects, the activator domain has a binding specificity to a receptor tyrosine kinase at the cell surface. In some embodiments, the binding of the activator to the tyrosine kinase receptor activates intracellular signaling pathways associated with cell survival. In some aspects, the activator domain has a binding specificity to a receptor that modulates/promotes tissue regeneration.

In some embodiments, the targeting domain serves to target the chimeric protein to a target cell or tissue while the activator domain serves to activate the intracellular signaling pathway associated with cell/tissue survival or tissue regeneration.

In some embodiments, the growth factor (e.g. IGF-1 variant) is engineered to reduce potency while retaining the ability to activate the cognate growth factor receptor. In some embodiments, wild type growth factors can be used as activator domains.

In some embodiments, the chimeric protein comprises an activator domain having a growth factor variant that is selected to give the chimeric protein at least an order of magnitude lower EC50 in damaged tissue than in healthy tissue. For example, the chimeric protein domain comprises a growth factor variant and has an EC50 in damaged tissue that is at least 10 times lower, at least 15 times lower, at least 20 times lower, at least 25 times lower, at least 30 times lower, at least 35 times lower, at least 40 times lower, at least 45 times lower, at least 50 times lower, at least 55 times lower, at least 60 times lower, at least 65 times lower, at least 70 times lower, at least 75 times lower, at least 80 times lower, at least 85 times lower, at least 90 times lower, at least 95 times lower, at least 100 times lower, at least 110 times lower than the EC50 in healthy tissue.

The targeting domain is generally used to target the chimeric proteins to a target cell. In some embodiments, the target cell is undergoing apoptosis. The binding of the targeting domain to its target molecule does not induce a significant biological effect in the target cell. The activator domain binds to a receptor on a cell surface. The binding of the activator domain to its receptor is intended to modulate a specific biological effect, such as, activate the intracellular signaling pathway associated with cell survival. In some embodiments, binding of the activator domain to its receptor is intended to positively regulate survival of the targeted cells or tissue. In particular, the activator domain of chimeric protein can promote survival signaling.

In some embodiments, the in vivo activity of the chimeric protein can be assessed by detecting signaling changes in molecules that are regulated by the activator domain, including but not limited to cell surface receptor phosphorylation status or downstream mediators such as phospho-AKT or phospho-ERK (as detected by flow cytometry, immunofluorescence, ELISA, phospho-labeling, Western analysis of treated tissues, or any other methodology known in the art.) In some embodiments, a chimeric protein functions in vivo if it induces a significant (e.g., at least 10%, at least 20%, at least 30%, at least 40%, at least 50% or more) change in the level, functional activity, or phosphorylation of the regulated molecule detected by the assay.

In some embodiments, the half-life modulator comprises a peptide that extends the half-life of the chimeric protein.

In some embodiments, the chimeric proteins are fusion proteins having a targeting polypeptide connected or linked to a half-life modulator and to an activator polypeptide. In some embodiments, the engineered proteins are chimeric proteins having a targeting polypeptide connected or linked to a half-life modulator and a growth factor or mutated growth factor.

### Activator Domain

The activator domain can be any polypeptide that detectably modulates the activity of a cellular network. In some embodiments, the activator domain is capable of activating signal transduction pathways by binding to a receptor at the surface a cell. In some embodiments, certain activator domains are growth factor polypeptides, or any agonist of the receptor. It will be apparent that such modulation may be an increase in the activity of the cellular network such as induction of proliferation of cells, induction of cell growth, promotion of cell survival and/or inhibition of apoptosis.

In some embodiments, the activator domain comprises a change in the amino acid sequence, the three-dimensional structure of the protein, and/or the activity of the protein, relative to the wild-type form of the protein.

In some embodiments, the activator domain comprises or consists of a growth factor having amino acid sequence modification relative to the wild-type growth factor (e.g. IGF-1) to decrease its binding to its natural receptor (e.g. IGF-1 receptor), to decrease its binding to binding proteins (e.g. IGF binding proteins) and/or decrease its activation of its natural receptor (e.g. IGF-1 receptor). In some embodiments, the activator domain is a growth factor having amino acid sequence modification that reduce (e.g., for about 1-5%, 5-10%, 10%-20%, about 20%-40%, about 50%, about 40%-60%, about 60%-80%, about 80%-90%, 90-95%) the binding to its natural receptor (e.g. IGF-1 receptor).

A growth factor polypeptide detectably modulates activation of a growth factor receptor. In some embodiments, the activator domain of the chimeric protein is a growth factor, variant or fragment thereof that retains at least about 0.01% of wild-type biological activity. In some embodiments, the activator domain of the chimeric protein is a growth factor, variant or fragment thereof that retain at least about 0.1%, at least about 1%, at least about 10%, of wild-type biological activity. In some embodiments, the activator domain of the chimeric protein is a growth factor, variant or fragment thereof that retains between about 0.01% to about 0.1% of wild-type biological activity. In some embodiments, the activator domain of the chimeric protein is a growth factor, variant or fragment thereof that retains between about 0.01% to about 1% of wild-type biological activity. In some embodiments, the activator domain of the chimeric protein is a growth factor, variant or fragment thereof that retains between about 0.01% to about 10% of wild-type biological activity. In some embodiments, the activator domain of the chimeric protein is a growth factor, variant or fragment thereof that retains between about 0.1% to about 1% of wild-type biological activity. In some embodiments, the activator domain of the chimeric protein is a growth factor, variant or fragment thereof that retains between about 0.1% to about 10% of wild-type biological activity. In some embodiments, the activator domain of the chimeric protein is a growth factor, variant or fragment thereof that retains between about 01% to about 10% of wild-type biological activity. Biological activity in some embodiments can be determined by measuring activation of the corresponding growth factor receptor in appropriate cells. In some embodiments, activation may be assessed, for example, by measuring phosphorylation of receptor kinase or downstream effector proteins, such as, but not limited to, AKT, S6, ERK, INK, mTOR, etc. Insulin-like growth factors (IGFs) and derivatives thereof

The insulin-like growth factors (IGFs) constitute a family of proteins having insulin-like and growth stimulating properties. The IGFs Human IGF-1 is a 70 amino acids basic peptide having the protein shown in SEQ ID NO: 1, respectively. IGF-1 and extracellular tyrosine kinase receptor (e.g. IGF-1 receptor) are important for cellular processes such as cell proliferation and survival. Binding of IGF-1 or variant thereof to the IGF-1 receptor stimulates kinase activity, leading to phosphorylation of multiple substrates, thereby initiating signaling cascades. The chimeric proteins disclosed herein can maintain the ability to signal through the extracellular receptor, for example IGF-1 receptor. The activator domain IGF-1 stimulates cell proliferation and survival through activation of the AKT pathway. Upon binding of IGF-I to the IGF-1 receptor, a tyrosine kinase, phosphorylates tyrosine residues on two major substrates, IRS-1 and Shc, which subsequently signal through the Ras/Raf and PI 3-kinase/AKT pathways.

The interaction of IGF-1 (and IGF-2) with the IGF-1 receptor is regulated by IGF binding Proteins (IGFBPs). All six IGFBPs (particularly IGFBP5) have been shown to inhibit IGF action, but in some instances a stimulatory effect has been observed. At least 99% of the IGF in the circulation is normally bound to IGFBPs.

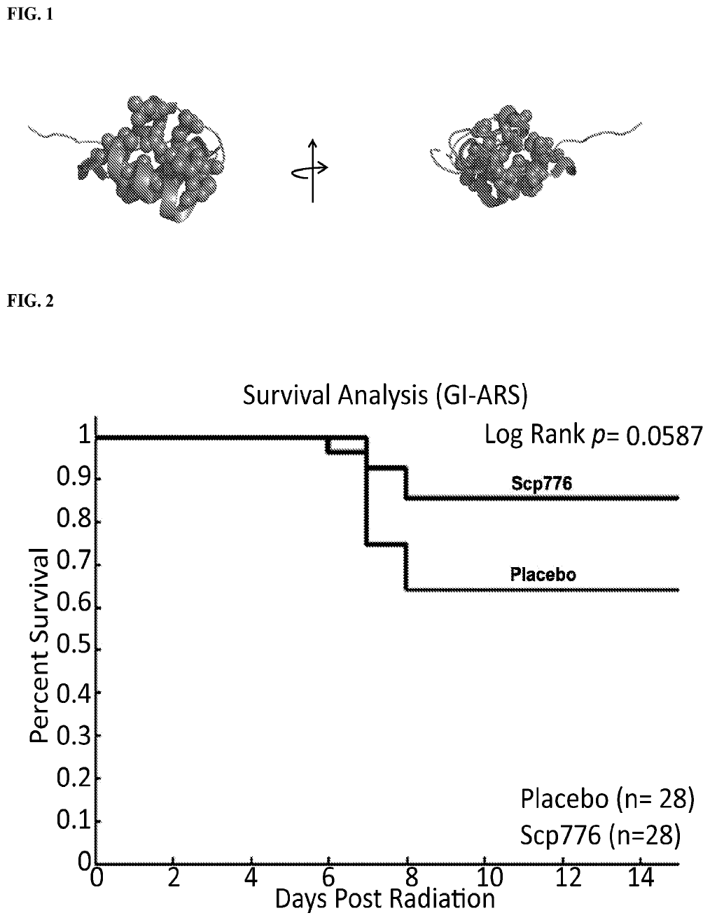

In some embodiments, the activator domain is a variant of the human IGF-1 or fragment thereof. In some embodiments, the variant of IGF-1 or fragment thereof is capable of maintaining selectivity to the IGF-1 receptor. FIG. 1 shows residues identified via application of an algorithm that considers solvent accessibility, hydrophobicity, and temperature factor. Represented by X are residue position that can be mutated. Collectively, these residues represent the hydrophobic core of IGF-1. Hydrophobic core positions of IGF-1 are depicted as blue spheres (pdb model 1H02) at FIG. 1.

In some embodiments, the IGF-1 variant is modified to reduce binding to IGF-1 binding proteins (IGFBPs) relative to wild-type IGF-1 while maintaining its ability to activate the AKT pathway. In some embodiments, the IGF-1 variant can activate the IGF-1 receptor with a decreased potency for non-target cells, as assessed by pAKT EC50. EC50 is defined as the concentration needed to achieve the half maximal level of pAKT signaling.

In some embodiments, the IGF-1 variant comprises a substitution at one or more of the tyrosine residues. In some embodiments, the IGF-1 variant comprises one or more substitutions at position Y24, Y31 and Y60.

In some embodiments, the IGF-1 variant can comprise a single tyrosine substitution at position Y31, or Y24, or Y60. In some embodiments, the IGF-1 variant can comprise a tyrosine substitution at positions Y24 and Y31, Y24 and Y60, Y31 and Y60, or Y24 and Y60. In some embodiments, the IGF-1 variant can comprise a tyrosine substitution at position Y24, Y31 and Y60.

In some embodiments, the tyrosine at position 24 can be substituted with alanine (Y24A), valine (Y24V), leucine (Y24L), glycine (Y24G), methionine (Y24M), serine (Y24S), asparagine (Y24N) or Glutamine (Y24Q). In some embodiments, the tyrosine at position 31 can be substituted with alanine (Y31A), valine (Y31V), leucine (Y31L), glycine (Y31G), methionine (Y31M), serine (Y31S), asparagine (Y31N) or Glutamine (Y31Q). In some embodiments, the tyrosine at position 60 can be substituted with alanine (Y60A), valine (Y60V), leucine (Y60L), glycine (Y60G), methionine (Y60M), serine (Y60S), asparagine (Y60N) or Glutamine (Y60Q).

In another exemplary embodiment, the IGF-1 variant can comprise one or more of the following substitutions, Y24L, Y31A, and Y60L relative to wild type IGF-1. For example, the IGF-1 variant can comprise the Y24L substitution and the Y31A substitution or the IGF-1 variant can comprise the Y24L substitution, the Y31A substitution and the Y60L. In some embodiments, one or more tyrosine residues (Y24, Y31, Y60 or combinations thereof) can be substituted for a short aliphatic amino acid. In some embodiments, one or more tyrosine residues (Y24, Y31, Y60 or combinations thereof) can be substituted for a polar amino acid. In some embodiments, one or more tyrosine residues (Y24, Y31, Y60 or combinations thereof) can be substituted for leucine, alanine, isoleucine, serine, threonine or any other amino acid.

In some embodiments, the glutamic acid at position 3 can be substituted with alanine, arginine, asparagine, cysteine, glutamine, glycine, histidine, isoleucine, leucine, lysine, methionine, phenylalanine, proline, serine, threonine, tryptophan, tyrosine or valine. In some embodiments, the glutamic acid at position 3 is replaced with a basic and/or aliphatic amino acid. In some embodiments, the glutamic acid at position 3 can be substituted with arginine (E3R) or lysine (E3K). In some embodiments, the IGF-1 variant comprises E3R substitution. In some embodiments, the IGF-1 variant comprises E3R substitution. In some embodiments, the activator domain has an amino acid sequence having SEQ ID NO: 4 or SEQ ID NO: 38.

In some embodiments, the IGF-1 variant comprises a substitution at the position 3 and 31. For example, the IGF-1 variant comprises E3R and Y31A substitutions or E3K and Y31A substitutions. In some embodiments, the activator domain has an amino acid sequence having SEQ ID NO: 2 or SEQ ID NO: 40. In some embodiments, the IGF-1 variant comprises a substitution at the position E3 and Y24. For example, the IGF-1 variant comprises E3R and Y24L substitutions or E3K and Y24L substitutions. In some embodiments, the activator domain has an amino acid sequence having SEQ ID NO: 25 or SEQ ID NO: 39. In some embodiments, the IGF-1 variant comprises a substitution at the position E3 and Y60. For example, the IGF-1 variant comprises E3R and Y60L substitutions or E3K and Y60L substitutions. In some embodiments, the activator domain has an amino acid sequence having SEQ ID NO: 26 or SEQ ID NO: 41. In some embodiments, the IGF-1 variant comprises a substitution at the position E3, Y24 and Y31. For example, the IGF-1 variant comprises E3R, Y24L, Y31A substitutions or E3K, Y24L, Y31A substitutions. In some embodiments, the activator domain has an amino acid sequence having SEQ ID NO: 27 or SEQ ID NO: 45. In some embodiments, the IGF-1 variant comprises a substitution at the position E3, Y24 and Y60. For example, the IGF-1 variant comprises E3R, Y24L and Y60L substitutions or E3K, Y24L and Y60L substitutions. In some embodiments, the activator domain has an amino acid sequence having SEQ ID NO: 28 or SEQ ID NO: 42. In some embodiments, the IGF-1 variant comprises a substitution at the position E3, Y31 and Y60. For example, the IGF-1 variant comprises E3R, Y31A and Y60L substitutions or E3K, Y31A and Y60L. In some embodiments, the activator domain has an amino acid sequence having SEQ ID NO: 29 or SEQ ID NO: 44. In some embodiments, the IGF-1 variant comprises a substitution at the position E3, Y24, Y31 and Y60. For example, the IGF-1 variant comprises E3R, Y24L, Y31A and Y60L substitutions or E3K, Y24L, Y31A and Y60L substitutions. In some embodiments, the activator domain has an amino acid sequence having SEQ ID NO: 30 or SEQ ID NO: 43.

In some embodiments, the activator domain is a derivative of the human IGF-1 comprising one or more of the following modifications: a N-terminal 13-residue extension (IGF-1 LONG), a deletion of amino acids 1-3 (Des-1-3), a substitution replacing Arg for a Glu at the 3 position of the polypeptide (E3R), no Arginine at position 37 (R37X), a deletion of amino acids 68-70 (3X), an N-terminal 13-residue extension and a substitution replacing Arg for a Glu at the 3 position of the wild-type polypeptide (LR3), substitutions of one or more of tyrosine residues Y24, Y31, Y60 or combinations thereof (e.g. Y24L, Y31A, Y60L substitutions or combinations thereof).

In some embodiments, the activator domain is variant of the human IGF-1 comprising a mutation (e.g. substitution, deletion) at one or more residues 24 to 37.

In some embodiments, the activator domain is a derivative of the human IGF-1 and comprises an N-terminal 13-residue extension (also referred as IGF-1 LONG, SEQ ID NO: 3), a mutation E3R (SEQ ID NO: 4) or a combination thereof (LONG E3R, also referred as LR3, SEQ ID NO: 6). In some embodiments, the IGF-1 variant comprises the E3R substitution, an N-terminal 13-residue extension, deletion of amino acids 1-3 ((Des1-3), SEQ ID NO: 5) or a combination thereof to decrease the binding of the activator domain to the IGF binding proteins which are present in the serum and other body fluid.

In some embodiments, the activator domain is a derivative of the human IGF-1 and comprises one or more of the following modifications: an N-terminal 13-residue extension (SEQ ID NO: 3), a deletion of amino acids 1-3 (SEQ ID NO: 5), a substitution replacing Arg for a Glu at the 3 position of the polypeptide (SEQ ID NO: 4), no Arginine at position 37 (R37X, SEQ ID NO: 7), a deletion of amino acids 68-70 (3X, SEQ ID NO: 8), or an N-terminal 13-residue extension and a substitution replacing Arg for a Glu at the 3 position of the wild-type polypeptide (SEQ ID NO: 6).

It is believed that the chimeric proteins that contain IGF-1 LONG, IGF-1 LONG E3R (referred to as IGF-1(LR3)) or IGF1 Des1-3, have decreased affinity for IGF binding proteins relative to wild-type IGF-1. In some embodiments, the IGF-1 variants of the chimeric proteins described herein can activate the signaling pathway while having a substantially decreased interaction with the IGF-1 binding proteins relative to wild-type IGF-1.

In some embodiments, the IGF-1 variant can be modified by glycosylation of one or more glyscosylation site present in the IGF-1 variant.

In some embodiments, the chimeric proteins that contain the IGF-1 variants described herein have a potency for non-target cells that is less than wild-type IGF-1 for non-target cells.

Certain activator domains that bind to growth factor receptors are provided herein in any of SEQ ID NOs: 1-8, 25-30, 38-45. Additional peptide sequence modifications can be included, such as variations, deletions, substitutions or derivatizations of the amino acid sequence of the sequences disclosed herein, so long as the peptide has substantially the same activity or function as the unmodified peptides. Notably, a modified peptide will retain activity or function associated with the unmodified peptide, the modified peptide will generally have an amino acid sequence “substantially homologous” with the amino acid sequence of the unmodified sequence.

In some embodiments, the IGF-1 variant can have an amino acid sequence having at least about 85%, at least about 90%, at least about 91%, at least about 92%, at least about 93%, at least about 94%, at least about 95%, at least about 97%, at least about 98% identity or at least about 99% identity to the amino acid sequence provided in any one of SEQ ID NOs: 1-8, 25-30, 38-45. In some embodiments, the IGF-1 variant can have an amino acid sequence having from about 85% to about 90%, from about 90% to about 95%, from about 95% to about 98%, from about 98% identity to about 99% identity to the amino acid sequence provided in any one of SEQ ID NOs: 1-8, 25-30, 38-45. In some embodiments, the IGF-1 variant can comprise 10, 20, 30, 40, 50, 60 or more consecutive amino acids of any one of amino acids in any one of SEQ ID NOs: 1-8, 25-30, 38-45. In some embodiments, the IGF-1 variant can have an amino acid sequence recited in any one of SEQ ID NOs: 1-8, 25-30, 38-45. In some embodiments, the IGF-1 variant can have an amino acid sequence recited in any one of SEQ ID NOs: 2-8, 25-30, 38-45. In some embodiments, the IGF-1 variant can have an amino acid sequence recited in SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 25, SEQ ID NO: 26, SEQ ID NO: 27, SEQ ID NO: 28, SEQ ID NO: 29, SEQ ID NO: 30, SEQ ID NO: 38, SEQ ID NO: 39, SEQ ID NO: 40, SEQ ID NO: 41, SEQ ID NO: 42, SEQ ID NO: 43, SEQ ID NO: 44 or SEQ ID NO: 45.

Suitable conservative substitutions of amino acids are known to those of skill in the art and may be made generally without altering the biological activity of the resulting molecule. Those of skill in this art recognize that, in general, single amino acid substitutions in non-essential regions of a polypeptide do not substantially alter biological activity.

In some embodiments, the activator domain comprises or consists of an amino acid sequence according to any of SEQ ID NOs: 1-8, 25-30, 38-45. In some embodiments, the activator domain comprises or consists of an amino acid sequence with an identity of at least 85%, at least 90%, at least 91%, at least 92%, at least 93%, at least 94%, at least 95%, at least 96%, at least 97%, at least 98%, at least 99% according to any of SEQ ID NOs: 1-8, 25-30, 38-45. In some embodiments, the chimeric proteins that contain the IGF-1 variants have a half maximal effective concentration (EC50) that is lower in damaged tissue than in healthy tissue. In some embodiments, the chimeric proteins that contain the IGF-1 variants have a half maximal effective concentration (EC50) that is at least 10 times lower, at least 15 times lower, at least 20 times lower, at least 25 times lower, at least 30 times lower, at least 35 times lower, at least 40 times lower, at least 45 times lower, at least 50 times lower, at least 55 times lower, at least 60 times lower, at least 65 times lower, at least 70 times lower, at least 75 times lower, at least 80 times lower, at least 85 times lower, at least 90 times lower, at least 95 times lower, at least 100 times lower, at least 110 times lower in damaged tissue than in healthy tissue.

In some embodiments, the chimeric proteins provided herein having such variant growth factors have a higher specificity to the damaged tissue targeted or apoptotic cells.

### Neuregulin (NRG) and Derivatives Thereof

As used herein the term “neuregulin” or NRG refers to a member of a family of structurally related proteins that are part of the EGF family of proteins. Multiple family members are generated by alternate splicing or by use of several cell type-specific transcription initiation sites. In general, they bind to and activate the erbB family of receptor tyrosine kinases (ErbB-2 (HER2), ErbB-3 (HER3), and ErbB-4 (HER4)), functioning both as heterodimers and homodimers.

In some embodiments, the activator domain is neuregulin, neuregulin Beta1, neuregulin alpha, neuregulin alpha1A, neuregulin 3, neuregulin beta2, fragment thereof or variants thereof. In some embodiments, the EGF-like domain of neuregulin-1 takes part in signal transduction by stimulating the ErbB3 receptor tyrosine kinase ErbB2.

In some embodiments, the neuregulin protein can bind and activate ErbB-2 (as part of a heterodimer with ErbB-3, ErbB-4) and include but not limited to all neuregulin isoforms, neuregulin EGF domain alone, polypeptides comprising neuregulin EGF-like domain, neuregulin mutants or derivatives, and any kind of neuregulin-like gene expression products that can activate erbb-2.

In some embodiments, the NRG variant can have an amino acid sequence having at least about 85%, at least about 90%, at least about 91%, at least about 92%, at least about 93%, at least about 94%, at least about 95%, at least about 97%, at least about 98% identity or at least about 99% identity to the amino acid sequence provided in any one of SEQ ID NOs: 31-37. In some embodiments, the NRG variant can have an amino acid sequence having from about 85% to about 90%, from about 90% to about 95%, from about 95% to about 98%, from about 98% identity to about 99% identity to the amino acid sequence provided in any one of SEQ ID NOs: 31-37. In some embodiments, the NRG variant can comprise 10, 20, 30, 40, 50, 60 or more consecutive amino acids of any one of amino acids in any one of SEQ ID NOs: 31-37. In some embodiments, the NRG variant can have an amino acid sequence recited in any one of SEQ ID NOs: 31-37. In some embodiments, the NRG variant can have an amino acid sequence recited in any one of SEQ ID NOs: 31-37. In some embodiments, the NRG variant can have an amino acid sequence recited in, SEQ ID NO: 31, SEQ ID NO: 32, SEQ ID NO: 33, SEQ ID NO: 34, SEQ ID NO: 35, SEQ ID NO: 36, or SEQ ID NO: 37.

In some embodiments, the variant of human neuregulin Beta1 comprises a G1S substitution. In some embodiments, the variant of human neuregulin Beta1 comprises or consists of SEQ ID NO: 32.

Suitable conservative substitutions of amino acids are known to those of skill in the art and may be made generally without altering the biological activity of the resulting molecule. Those of skill in this art recognize that, in general, single amino acid substitutions in non-essential regions of a polypeptide do not substantially alter biological activity.

In some embodiments, the activator domain comprises or consists of an amino acid sequence according to any of SEQ ID NOs: 31-37. In some embodiments, the activator domain comprises or consists of an amino acid sequence with an identity of at least 85%, at least 90%, at least 91%, at least 92%, at least 93%, at least 94%, at least 95%, at least 96%, at least 97%, at least 98%, at least 99% according to any of SEQ ID NOs: 31-37.

In some embodiments, the chimeric proteins that contain the IGF-1 variants have a half maximal effective concentration (EC50) that is lower in damaged tissue than in healthy tissue. In some embodiments, the chimeric proteins that contain the IGF-1 variants have a half maximal effective concentration (EC50) that is at least 10 times lower, at least 15 times lower, at least 20 times lower, at least 25 times lower, at least 30 times lower, at least 35 times lower, at least 40 times lower, at least 45 times lower, at least 50 times lower, at least 55 times lower, at least 60 times lower, at least 65 times lower, at least 70 times lower, at least 75 times lower, at least 80 times lower, at least 85 times lower, at least 90 times lower, at least 95 times lower, at least 100 times lower, at least 110 times lower in damaged tissue than in healthy tissue.

In some embodiments, the chimeric proteins provided herein having such variant growth factors have a higher specificity to the damaged tissue targeted or apoptotic cells.

### Target Molecules

In some aspects, target molecules are exposed or enriched on the exterior of a target cell. In some embodiments, the target molecule is associated with a damaged cell, early apoptotic or apoptotic cell, the target molecule being intracellular in a viable or undamaged cell and being exposed to the extracellular space in a damaged cell. Such molecules include, for example, molecules that are exposed in cells that undergo necrosis (such as DNA) or apoptosis (e.g., phosphatidylserine), myosin (including the tissue type-specific subtypes thereof), ICAM-1 or P-selectin. Yet in other embodiments, the target molecule is a molecule that is present or enriched at the surface of a diseased or dysfunctional cell or tissue as compared to the level detected in a healthy or functional cell or tissue. In some embodiments, the target cell is not a tumor or cancerous cell.

Cells are bounded by a plasma membrane (or cell membrane) comprising a lipid bilayer. The cell membrane may be considered to have a surface facing the cytosol (cytosolic side or interior of the cell) and a surface facing the exterior of the cell, or the extracellular space. Trans-bilayer movement of anionic phospholipids from the inner to the outer leaflet of the plasma membrane occurs during apoptosis. The anionic phospholipid-binding protein, such as Annexin A5, synaptotagmin I or lactadherin can be used to detect the presence of phosphatidylserine on the outer leaflet of the cell membrane. Phosphatidylserine is a phospholipid, that is usually restricted to the cytosolic side of the membrane in viable or undamaged cells, and that becomes exposed on the outer cell surface or to the extracellular space in damaged cells or apoptosis.

In some embodiments, the target molecule is a molecule that is detected at a level that is significantly higher (e.g., at least 1.5 higher, at least 2-fold higher, at least 3-fold higher, at least 4-fold higher, at least 5-fold higher) following ionizing radiation than in a cell of the same tissue that has not undergone an ionizing radiation.

### Targeting Domain

In some embodiments, the targeting domain has a specific binding affinity to a target molecule associated with a tissue. In some embodiments, the targeting domain has a specific binding affinity for a target molecule presented on the surface of early apoptotic cells. The targeting domain may be any polypeptide sequence that serves this function. In some embodiments, binding of the targeting domain to the target molecule does not have or does not modulate a biological activity. As used herein, “biological activity” refers to a defined, known activity performed by exposure of a molecule to a domain of the protein.

In some embodiments, the targeting domain can be a non-antibody polypeptide, fragment thereof or variant thereof having a binding affinity to the target molecule. Yet in other embodiments, the targeting polypeptide domain comprises one or more antibody variable regions (e.g. scFv).

### Annexin A5 and Variants Thereof

In some embodiments, the targeting domain comprises annexin, a variant thereof or a fragment thereof. The term “annexin” refers to any protein capable of binding to phospholipids, especially phosphatidylserine (PS), and member of the annexin family. In some embodiments, the annexin is Annexin A5 but other annexins can equally be used. In some embodiments, the targeting domain is human Annexin A5, a functional fragment thereof, or a variant thereof. A variant of Annexin A5 comprises at least one amino acid in at least one position in which this amino acid is not found in the parent wild type Annexin A5 polypeptide (SEQ ID NO: 9). The annexin variants according may comprise one or more amino acid substitutions, deletions, additions, or combinations thereof wherein the amino acid substitutions, deletions, or additions do not substantially affect the ability of the Annexin A5 variant of the chimeric protein to bind to at least one phospholipid, such as PS. In some embodiments, the Annexin A5 variant can have an amino acid sequence having at least about 85%, at least about 90%, at least about 95%, at least about 98% identity or at least about 99% identity to the amino acid sequence provided in SEQ ID NO: 9. In some embodiments, the Annexin A5 variant can comprise 50, 80, 100, 110, 200, 300, or more consecutive amino acid having at least about 85%, at least about 90%, at least about 95%, at least about 98% identity or at least about 99% identity to the amino acids in SEQ ID NO: 9.

In some embodiments, the variant of Annexin A5 is modified to substitute cysteine at position 315 (corresponding to C316) with serine or alanine to reduce dimer formation. For example, the cysteine can be substituted to an alanine or a serine. As used herein, the term “corresponding to” is used to designate the position/identity of an amino acid residue in a polypeptide (e.g., Annexin A5). Those of ordinary skill will appreciate that, for purposes of simplicity, a canonical numbering system (based on wild-type Annexin A5) is utilized herein, so that an amino acid “corresponding to” a residue at position 316, for example, need not actually be the 316th amino acid in a particular amino acid chain but rather corresponds to the residue found at position 316 in a for example Annexin A5 before the post-translational removal of the N-terminal methionine; those of ordinary skill in the art readily appreciate how to identify corresponding amino acids. In particular, it is noted that the amino acid sequence of wild-type Annexin A5 (SEQ ID NO: 9) do not start with a Methionine as the Methionine residue is cleaved during processing.

In some embodiments, the variant of Annexin A5 has an amino acid sequences that has been mutated to reduce internalization of Annexin A5 or the chimeric protein comprising the variant of Annexin A5 into a cell while maintaining binding affinity to phosphatidylserine (PS). In some embodiments, the variant of Annexin A5 or the chimeric protein comprising the variant of Annexin A5 has a binding affinity to phosphatidylserine and is not internalized into a cell or is internalized at a slower rate than wild-type annexin A5. In some embodiments, the targeting domain is a non-internalizing variant of Annexin A5, (also referred as ni-Annexin A5 or ni-AnxV, SEQ ID NO: 11). In some embodiments, the variant of Annexin A5 has an amino acid set forth in SEQ ID NO: 10. In some embodiments, the non-internalizing mutant of Annexin A5 has an amino acid sequence having at least about 85%, at least about 90%, at least about 95%, at least about 98% identity or at least about 99% identity to the amino acid sequence provided in SEQ ID NO: 11. In some embodiments, the non-internalizing mutant of Annexin A5 can have an amino acid sequence having from about 85% to about 90%, from about 90% to about 95%, from about 95% to about 98%, from about 98% to about 99% identity to the amino acid sequence provided in SEQ ID NO: 11. In some embodiments, the Annexin A5 variant can comprise 50, 80, 100, 110, 200, 300, or more consecutive amino acid of any one of amino acids in SEQ ID NO: 11. Any variation of Annexin A5 that results in substantially no internalization is envisioned.

It should be appreciated that the non-internalizing variant of annexin A5 can confer an extended half-life to the chimeric protein as compared to a chimeric protein that contains wild-type A5. In some embodiments, the variants of annexin A5 that results in substantially no internalization, or chimeric proteins containing variants of annexin A5 that results in substantially no internalization, can have an extended half-life of 1.1 to 1.2, 1.1 to 1.3, 1.1. to 1.4, 1.1 to 1.5, 1.1 to 1.6, 1.1 to 1.7, 1.1 to 1.8, 1.1 to 1.9, 1.1 to 2 or greater as compared to wild-type annexin A5, or chimeric proteins containing wild-type annexin A5.

The terms “non-internalizing” and “substantially no internalization,” as used herein, refer to a lack of internalization of a substantial amount of the chimeric protein disclosed herein. For example, the phrase “substantially no internalization” will be understood as less than 50% of the chimeric protein being internalized by a cell to which the chimeric protein is bound, or less than 25% of the chimeric protein being internalized by a cell to which the chimeric protein is bound, or less than 10% of the chimeric protein being internalized by a cell to which the chimeric protein is bound, or less than 5% of the chimeric protein being internalized by a cell to which the chimeric protein is bound, or less than 3% of the chimeric protein being internalized by a cell to which the chimeric protein is bound, or less than 1% of the chimeric protein being internalized by a cell to which the chimeric protein is bound.

In some embodiments, the non-internalizing mutant of Annexin A5 can have an amino acid sequence having at least about 85%, at least about 90%, at least about 95%, at least about 98% identity or at least about 99% identity to Annexin A5 modified to substitute cysteine at position 315 (corresponding to C316) with serine or alanine.

In some embodiments, Annexin A5 or Annexin A5 variants (for example having a substitution at C316, D143 and/or E227) are modified to comprise one or more of the following substitutions R62A, K69A, K100A, E137A, D138G, N159A, L313E (corresponding to R63A, K70A, K101A, E138A, D139G, N160A, L314E). For example, Annexin A5 having SEQ ID NO: 9 can be modified to have C315A or C315S substitution (corresponding to C316A or C316S relative to wild type Annexin A5) and one or more of the following substitutions R62A, K69A, K101A, E137A, D138G, N159A, L313E (corresponding to R63A, K70A, K101A, E138A, D139G, N160A, L314E relative to wild type Annexin A5).

In some embodiments, Annexin A5 (SEQ ID NO: 9) or Annexin A5 variants (for example having a substitution at C316, D143 and/or E227) are modified to comprise one or more of the following substitutions R62A, K69A, K100A, E137A, D138G, N159A, D143N, E227A, C315S or C315A (corresponding to R63A, K70A, K101A, E138A, D139G, D144N, N160A, E228A, C316S or C316A relative to wild type Annexin A5).

In some embodiments, the targeting domain is Annexin A5 which has been engineered to have R63A, K70A, K101A, E138A, D139G, N160A and C316A or C316S substitutions relative to wild type Annexin A5. For example, the targeting domain can have the amino acid sequence of SEQ ID NO: 10.

In some embodiments, the Annexin A5 variant comprises one or more, two, or two or more substitutions in different regions, in order to further decrease the internalization of the annexin in a cell. For example, the Annexin A5 variants may comprise R62A and K69A, R62A and K100A, R62A and E137A, R62A and D138G, R62A and N159A, R62A and K69A and K100A, R62A and K69A and E137A, etc . . . .

The annexin variants according may further comprise one or more amino acid substitutions, deletions, or additions, wherein the amino acid substitutions, deletions, or additions do not substantially affect the ability of the Annexin A5 variant of the chimeric protein to bind to at least one phospholipid, such as PS.

Native polypeptide can be used as targeting domains. It will be apparent, however, that portions of such native sequences and polypeptides having altered sequences may also be used, provided that such polypeptides retain the ability to bind the target molecule with an appropriate binding affinity (Kd) as described in more details below.

### Antibody Targeting Domain:

In some embodiments, an anti-phosphatidylserine antibody can be used as a targeting domain. As used herein, term “antibody” includes but is not limited to: (i) a Fab fragment, a monovalent fragment consisting of the VL, VH, CL and CHI domains; (ii) F(ab)2 and F(ab′)2 fragments, a bivalent fragment comprising two Fab fragments linked by a disulfide bridge at the hinge region; (iii) a Fd fragment consisting of the VH and CHI domains; (iv) a scFv fragment consisting of the VL and VH domains of a single arm of an antibody, (v) a dAb fragment which consists of a VH domain; and (vi) an isolated complementarity determining region (CDR). Such antibodies may be produced from intact antibodies using methods known in the art, or may be produced recombinantly, using standard recombinant DNA and protein expression technologies.

### Binding of Targeting Domain

In some embodiments, the chimeric protein binds to the target molecule with a K_{d} of less than 10⁻⁶ M, preferably less than 10⁻⁷ M, 10⁻⁸ M, 10⁻⁹ M or 10⁻¹⁰ M.

### Half-Life Modulator

One skilled in the art would appreciate that proteins used in therapeutic applications may not exhibit optimal serum half-lives due to their relatively low molecular weight. In some therapeutic applications, it may therefore be desirable to extend the half-life of the proteins. In some embodiments, to achieve accumulation of the chimeric protein to the diseased injured or damaged area of an organ, the chimeric protein is conjugated operatively associated or fused with a half-life modulator. Preferably, the half-life modulator is non-immunogenic peptide.

For example, short half-life is the most limiting attribute of wild-type growth factors as therapeutics. Intravenous administered IGF-1 has a serum half-life in humans of less than 1 hour. The extended half-life of chimeric proteins disclosed herein compared to IGF-1, for example, allows for 1) equivalent efficacy with less frequent dosing; 2) equivalent exposure at a lower dose; 3) lower Cmax at an equivalent exposure level, reducing the risk of Cmax-related toxicity.

In some embodiments, the half-life modulators can increase the in vivo half-life of the chimeric proteins. For example, the half-life of the chimeric proteins comprising the half-life modulator is about 1 hour, 2 hour, 3 hours, 4 hours, 5 hours, 6 hours or greater. For example, the half-life of the chimeric proteins can be about 8 hours or more when tested in cynomolgus monkey. In some embodiments, the half-life of the chimeric proteins comprising the half-life modulator is about 24 hours, or greater. In some embodiments, the half-life of the chimeric proteins comprising the half-life modulator is about a week or greater.

In some embodiments, the half-life modulator is non-immunogenic in humans.

In some embodiments, the half-life modulator is a peptide that interacts with cellular machinery that promote evasion of lysosomal degradation pathways (e.g. —FcRn receptor-mediated recycling).

In some embodiments, the half-life modulator is designed to extend the half-life of the chimeric protein through binding to serum components such as Human Serum Albumin (HSA). HSA is the most abundant protein in the blood and has a demonstrated safety in humans.

In some embodiments, the half-life modulator is a HSA variant. In some embodiments, the half-life modulator comprises at least 100 consecutive amino acids that are at least 70%, 80%, 85%, 90%, 91%, 92%, 93%, 94%, 95%, 96%, 97%, 98%, or 99% identical to wild type human serum albumin amino acid sequence (wtHSA, SEQ ID NO: 12). In some embodiments, the half-life modulator comprises at least 200 consecutive amino acids that are at least 70%, 80%, 85%, 90%, 91%, 92%, 93%, 94%, 95%, 96%, 97%, 98%, or 99% identical to wild type human serum albumin amino acid sequence. In some embodiments, the half-life modulator comprises at least 300 consecutive amino acids that are at least 70%, 80%, 85%, 90%, 91%, 92%, 93%, 94%, 95%, 96%, 97%, 98%, or 99% identical to wild type human serum albumin amino acid sequence. In some embodiments, the half-life modulator comprises at least 400 consecutive amino acids that are at least 70%, 80%, 85%, 90%, 91%, 92%, 93%, 94%, 95%, 96%, 97%, 98%, or 99% identical to wild type human serum albumin amino acid sequence. In some embodiments, the half-life modulator comprises at least 500 consecutive amino acids that are at least 70%, 80%, 85%0, % 91%, 92%, 93%, 94%, 95%, 96%, 97%, 98%, or 99% identical to wild type human serum albumin amino acid sequence.

In some embodiments, the HSA variant can have one of more of the following substitutions:
cysteine C58 can be substituted, for example, with a serine (C58S),
lysine K420 can be substituted for example, with a glutamic acid (K420E),
asparagine N527 can be substituted for example, with a glutamine (N527Q),
glutamic acid E505 can be substituted for example, with a glycine G (E505G),
valine V547 can be substituted for example, with an alanine (V547A),
asparagine N527 can be substituted for example, with a Glutamine (N527Q).

In some embodiments, the HSA variant can have amino acids 26-609 and have one of more of the following substitutions:
cysteine C58 can be substituted for example, with a serine (C58S),
lysine K420 can be substituted for example, with a glutamic acid (K420E),
asparagine N527 can be substituted for example, with a glutamine (N527Q),
glutamic acid E505 can be substituted for example, with a glycine G (E505G),
valine V547 can be substituted for example, with an alanine (V547A),
asparagine N503 and/or N527 can be substituted for example, with an Glutamine (N503Q and/or N527Q).

In some embodiments, the HSA variant (referred herein as mHSA) has the following substitutions: C34S, N503Q (SEQ ID NO: 13). In some embodiments, the HSA variant (referred herein as mHSA7) has the following substitutions C34S, N503Q, E505G and V547A (SEQ ID NO: 14). In some embodiments, the HSA variant has amino acids 26-609 and the following substitutions C58S and N527Q (SEQ ID NO: 15).

In some embodiments, the asparagine at position 503 and/or 527 of HSA, which may be deamidated and decrease half-life, can be removed by the N503Q substitution and/or the N527Q. In some embodiments, the cysteine C34 of HSA may be substituted to serine or alanine (S or A) to remove the free cysteine and minimize alternate disulfide-bond formation. In some embodiments, the half-life modulator is a modified version of the domain III (mHSA_dIII) of a modified HSA with the N503Q substitution and an additional terminal glycine. Such a modified version retains the HSA property of binding to FcRn and increased serum half-life.

In some embodiments, the half-life modulator is Fc domain of an antibody or a single chain constant fragment. In some embodiments, the half-life modulator comprises Fc regions of an immunoglobulin molecule (e.g. IgG). In some embodiments, the half-life modulator comprises at least 100 consecutive amino acids that are at least 70%, 80%, 85%, 90%, 91%, 92%, 93%, 94%, 95%, 96%, 97%, 98%, or 99% identical to a human Fc amino acid sequence. The Fc domain of an antibody has a natural capability to bind FcRn, resulting in an extended half-life. In some embodiments, the Fc domain of an antibody is engineered not to bind Fc(gamma)R. In an exemplary embodiment, the Fc domain is engineered to substitute N297 with Q (N297Q variant). In some embodiments, the half-life modulator is a monomeric variant form of Fc (scFc). For example, the subset of IgG heavy chain which naturally dimerizes to form Fc is hinge-CH2-CH3. In some embodiments, the Fc domain is engineered to form a single chain by linking the hinge-CH2-CH3 with a flexible linker such as GGGGSGGGGSGGGGSGGGGS (SEQ ID NO: 16) to create a hinge-CH2-CH3-linker-hinge-CH2-CH3 chain. In an exemplary embodiment, the single chain Fc (scFc) is engineered to substitute N297 with Q and C220 with S (N297Q, C220S).

In some embodiments, the half-life modulator is a single chain variable fragment (scFv) of an antibody targeted to albumin or other circulating protein. In some embodiments, the half-life modulator comprises an amino acid sequence that is at least 70%, 80%, 85%, 90%, 91%, 92%, 93%, 94%, 95%, 96%, 97%, 98%, or 99% identical to scFv amino acid sequence directed to a specific antigen, such as, but not limited to, albumin. In some embodiments, the half-life modulator comprises at least 50, at least 100, at least 150, at least 200, at least 250 consecutive amino acids that are at least 70%, 80%, 85%, 90%, 91%, 92%, 93%, 94%, 95%, 96%, 97%, 98%, or 99% identical to a scFv amino acid sequence directed to a specific antigen, such as, but not limited to, albumin.

In some embodiments, the half-life modulator is transferrin such as human transferrin (Tf, SEQ ID NO: 17). In some embodiments, the half-life modulator comprises an amino acid sequence that is at least 70%, 80%, 85%, 90%, 91%, 92%, 93%, 94%, 95%, 96%, 97%, 98%, or 99% identical to human transferrin amino acid sequence. In some embodiments, the half-life modulator comprises at least 100, at least 200, at least 300, at least 400, at least 500, at least 600, at least 650 consecutive amino acids that are at least 70%, 80%, 85%, 90%, 91%, 92%, 93%, 94%, 95%, 96%, 97%, 98%, or 99% identical to a human transferrin amino acid sequence.

In some embodiments, the half-life modulator comprises at least 100 consecutive amino acids that are at least 70%, 80%, 85%, 90%, 91%, 92%, 93%, 94%, 95%, 96%, 97%, 98%, or 99% identical to wild type human alpha-fetoprotein amino acid sequence (AFP, SEQ ID NO: 18). In some embodiments, the half-life modulator comprises at least 100 consecutive amino acids that are about 70%, 80%, 85%, 90%, 91%, 92%, 93%, 94%, 95%, 96%, 97%, 98%, or 99% identical to wild type human alpha-fetoprotein (AFP) amino acid sequence. In some embodiments, the N-linked glycosylation site of the AFP is removed by the N251Q substitution.

In some embodiments, the half-life modulator comprises at least 100 consecutive amino acids that are at least 70%, 80%, 85%, 90%, 91%, 92%, 93%, 94%, 95%, 96%, 97%, 98%, or 99% identical wild-type vitamin D-binding protein amino acid sequence (VDBP, SEQ ID NO: 19). In some embodiments, the half-life modulator comprises at least 100 consecutive amino acids that are about 70%, 80%, 85%, 90%, 91%, 92%, 93%, 94%, 95%, 96%, 97%, 98%, or 99% identical wild-type vitamin D-binding protein (VDBP) amino acid sequence. In some embodiments, the N-linked glycosylation site of the VDBP can be removed by the N288Q or N288T substitution.

In some embodiments, the half-life modulator comprises at least 100 consecutive amino acids that are at least 70%, 80%, 85%, 90%, 91%, 92%, 93%, 94%, 95%, 96%, 97%, 98%, or 99% identical to wild-type human transthyretin amino acid sequence (TTR, SEQ ID NO: 20). In some embodiments, the half-life modulator comprises at least 100 consecutive amino acids that are about 70%, 80%, 85% 90%, 91%, 92%, 93%, 94%, 95%, 96%, 97%, 98%, or 99% identical to wild type human transthyretin (TTR) amino acid sequence. In some embodiments, the transthyretin is modified to remove the N118 N-glycosylation site. In some embodiments, the half-life modulator is a monomeric form of TTR.

In some embodiments, the half-life modulator comprises at least 100 consecutive amino acids that are at least 70%, 80%, 85%, 90%, 91%, 92%, 93%, 94%, 95%, 96%, 97%, 98%, or 99% identical to a PASylation amino acid sequence. PASylation are proline-, alanine-, and/or serine-rich sequences that mimic PEGylation (see WO/2008/155134). In some embodiments, the half-life modulator comprises at least 100 consecutive amino acids that are about 70%, 80%, 85%, 90%, 91%, 92%, 93%, 94%, 95%, 96%, 97%, 98%, or 99% identical to a PASylation amino acid sequence. PASylation are proline-, alanine-, and/or serine-rich sequences that mimic PEGylation. Polypeptide stretches of proline, alanine, and/or serine form semi-structured three-dimensional domains with large hydrodynamic radius, thereby reducing clearance of fusion proteins. In some embodiments, the PASylation amino acid sequence is about 200, 300, 400, 500 or 600 amino acids long. For example, the PASylation is a 20 times repeat of the amino acid sequence ASPAAPAPASPAAPAPSAPA (SEQ ID NO: 21).

In some embodiments, the half-life modulator comprises the attachment of polyethylene glycol (PEG) chain or chains to the fusion proteins through chemical attachment either to the N- and/or C-terminus and/or to an amino acid side chain (e.g., PEG-maleimide attachment to cysteines). PEG chains form semi-structured three-dimensional domains with large hydrodynamic radius, thereby reducing clearance of fusion proteins.

In some embodiments, the half-life modulator comprises at least 100 consecutive amino acids that are at least 70%, 80%, 85%, 90%, 91%, 92%, 93%, 94%, 95%, 96%, 97%, 98%, or 99% identical to an albumin-binding domain human antibody (albudAb) amino acid sequence (SEQ ID NO: 22). In some embodiments, the half-life modulator comprises at least 100 consecutive amino acids that are about 70%, 80%, 85%, 90%, 91%, 92%, 93%, 94%, 95%, 96%, 97%, 98%, or 99% identical to an albumin-binding domain human antibody (albudAb) amino acid sequence. Albumin-binding domain antibodies can increase the fusion protein half-life by binding non-covalently to serum albumin (see WO2008/096158). In some embodiments, the albumin-binding domain human antibody is engineered to remove the C-terminal arginine to remove the Lys-Arg Kex2 protease site.

Representative such half-life modulators include those recited in any one of SEQ ID NOs: 12-15, 17-22.

In some embodiments, the half-life modulators can be modified to substitute the cysteine residues to serine or alanine residues to reduce the ability to form disulfide bonds.

In some embodiments, the targeting domain and activator domain can be joined via a half-life modulator. Accordingly, the half-life modulator can have two termini, an N-terminus and a C-terminus. In some embodiments, the half-life modulator is joined at one terminus via a peptide bond to the targeting polypeptide domain and is joined at the other terminus via a peptide bond to the activator domain. In certain embodiments, the half-life modulator is joined at the N-terminus to the C-terminus of the targeting polypeptide domain and at the C-terminus to the N-terminus of the activator domain. In other embodiments, the half-life modulator is joined at the C-terminus to the targeting polypeptide domain and at the N-terminus to the activator domain. Yet, in other embodiments, the half-life modulator is joined at one of the termini of the chimeric protein. For example, in some embodiments, the half-life modulator is joined at the C-terminus to the N-terminus of the activator domain. In other embodiments, the half-life modulator is joined at the N-terminus to the C-terminus of the targeting domain. In other embodiments, the half-life modulator can be joined at the N-terminus to the C-terminus of the activator domain. Yet in other embodiments, the half-life modulator can be joined at the N-terminus to the C-terminus of the targeting domain.

### Peptide Linkers

In some embodiments, the activator domain, half-life modulator, and targeting domain are linked by peptide linker (e.g., from 2 to 40, 2-50, 2-100 amino acid residues) such that upon target recognition and engagement by the targeting domain, the presentation of the activator domain is optimized for binding to and activation of extracellular receptors on the surface of cells that present the target at a given surface density (e.g. −5×10² molecules/1,000 Å2).

Targeted delivery of the activator domain for example IGF-1 for the activation of receptors on cells or tissues displaying a specific target requires appropriate presentation of both the activator domain and the targeting domain. In some embodiments, the flexibility of the linker is optimized for proper geometry of the engaged chimeric protein. Some of the principal determinants of the geometric constraints are the distances from the cell surface for the target and the receptor.

Additional optimization can be driven by the relative number of receptors and target molecules. At high ratios of Receptor:Target molecule, the engagement of both domains is reaction-limited. When the target molecule is more abundant than the receptor, the occupancy of both domains is diffusion-limited. Under the reaction-limit, optimal delivery of the activator domain is attained via short and rigid linkers. Under the diffusion limit, long and flexible linkers allow the activator domain to access a larger surface area. For cells with complex shapes (i.e. —bodies and neuronal processes) and receptor distributions, appropriate design of linker flexibility can enable precise targeting to sub-cellular regions.

In some embodiments, the peptide linker is present at the N-terminus, at the C-terminus or at both the N-terminus and the C-terminus of the half-life modulator at one or both ends. Suitable short connector polypeptides for use at the N-terminal end of the linker include, for example, dipeptides such as -Gly-Ser- (GS), -Gly-Ala- (GA) and -Ala-Ser- (AS). Suitable peptide linkers for use at the C-terminal end of the linker include, for example, dipeptides such as -Leu-Gln- (LQ) and -Thr-Gly- (TG). In some embodiments, the peptide linkers are longer than 2 amino acids. For example, the peptide linkers are 5, 6, 7, 8, 9, 10, 11, 12, 13, 14, 15, 16, 17, 18, 19 or 20 amino acids long or longer. In some embodiments, the peptide linkers are 20 or more 30 or more, 40 or more, 50 or more, 60 or more, 70 or more, 80 or more, 90 or more, 100 or more amino acids long. Preferably, such peptide linkers are flexible (for example glycine-rich) or structured (e.g., alpha-helix rich). In some embodiments, the linker comprises or consist of amino acids -Gly-Ser-Gly-Gly-Gly-Ser-Gly (SEQ ID NO: 23).

It will be apparent that elements in addition to those described above may optionally be included in the proteins provided herein. Such elements may be present for a variety of purposes, including to facilitate expression, preparation or purification of the chimeric protein, or to perform targeting functions.

### Representative Chimeric Protein

In some embodiments, representative chimeric proteins comprise (from N-terminal to C-terminal):
(a) a targeting polypeptide domain comprising or consisting of a non-internalizing human annexin V variant (e.g., comprising or consisting of amino acids 2-320 of wt human Annexin 5 and a substitution at C316, R63, K40, K101, E138, D139, N160);
(b) a linker peptide (e.g., -Gly-Ser-Gly-Gly-Gly-Ser-Gly);
(c) a half-life modulator (e.g., HSA variant comprising or consisting of amino acids 26-609 of wt human HSA and comprising substitution at C58 and N527);
(d) linker peptide (e.g., -Gly-Ser-Gly-Gly-Gly-Ser-Gly);
(e) an activator domain comprising or consisting of an IGF-1 variant (e.g., comprising a substitution at E3 and Y31.

In some embodiments, the chimeric protein comprises or consists of IGF1(E3R/Y31A)_lk7_HSA26-609(C58S/N527Q)_lk7_AnxV2-320(R63A/K70A/K101A/E138A/D139G/N160A/C316A) (also referred herein as IGF1 (E3R/Y31A)_lk7__mHSA_lk7_ni-AnxV or scp776). In some embodiments, the chimeric protein has an amino acid sequence as set forth in SEQ ID NO: 24.

In some embodiments, the chimeric protein comprises or consists of Nrg1a_lk7_mHSA_lk7_ni-AnxV (also referred herein as scp757).

In some embodiments, the chimeric protein comprises or consists of Nrg1b (G1S)_lk7_mHSA_lk7_ni-AnxV (also referred herein as scp767).

### Nucleic Acids

Provided herein are polynucleotides encoding the chimeric proteins that may be in the form of RNA or in the form of DNA, which DNA includes cDNA and synthetic DNA. The DNA may be double-stranded or single-stranded. The coding sequences that encode the variants of the present disclosure may vary as a result of the redundancy or degeneracy of the genetic code.

### Pharmaceutical Compositions

Pharmaceutical compositions comprising a therapeutically effective amount of at least one chimeric protein as described herein, together with at least one physiologically acceptable carrier, are provided. Such compositions may be used for treating patients who are suffering from, or at risk for, tissue damage, in order to prevent tissue damage, or to repair or regenerate damaged tissue.

As used herein, the term “physiologically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the chimeric protein is administered. Physiologically acceptable carriers can be sterile liquids, such as water and oils, including those of petroleum, animal, vegetable or synthetic origin (e.g., peanut oil, soybean oil, mineral oil, or sesame oil). Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water and ethanol. The composition, if desired, can also contain minor amounts of wetting or emulsifying agents, or pH buffering agents.

Pharmaceutical compositions may be formulated for any appropriate manner of administration, including, for example, parenteral, intranasal, topical, oral, or local administration, such as by a transdermal means for prophylactic and/or therapeutic treatment. These compositions can take any of a variety of well-known forms that suit the mode of administration, such as solutions, suspensions, emulsions, tablets, pills, capsules, powders, aerosols and sustained-release formulations. The composition can be formulated as a suppository, with traditional binders and carriers. Oral formulation can include standard carriers such as pharmaceutical grades of mannitol, lactose, starch, magnesium stearate, sodium saccharine, cellulose, magnesium carbonate, etc. Examples of suitable pharmaceutical modes of administration and carriers are described in “Remington: The Science and Practice of Pharmacy,” A. R. Gennaro, ed. Lippincott Williams & Wilkins, Philadelphia, Pa. (21.sup.st ed., 2005).

The pharmaceutical compositions provided herein can be administered parenterally (e.g., by intravenous, intramuscular, or subcutaneous injection), or intra-nasally or intra-thecally or by oral ingestion or by topical application. For parenteral administration, the chimeric protein can either be suspended or dissolved in the carrier. A sterile aqueous carrier is generally preferred, such as water, buffered water, saline or phosphate-buffered saline. In addition, sterile, fixed oils may be employed as a solvent or suspending medium. For this purpose, any bland fixed oil may be employed, including synthetic mono- or diglycerides. In addition, fatty acids such as oleic acid find use in the preparation of injectable compositions. Pharmaceutically acceptable auxiliary substances may also be included to approximate physiological conditions, such as pH adjusting and buffering agents, tonicity adjusting agents, dispersing agents, suspending agents, wetting agents, detergents, preservatives, local anesthetics and buffering agents.

In some embodiments, the pharmaceutical composition is formulated for intravenous administration to a patient (e.g., a human). Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a sealed (e.g., hermetically sealed) container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In some embodiments, compositions intended for oral use may be presented as, for example, tablets, troches, lozenges, aqueous or oily suspensions, dispersible powders or granules, emulsion, hard or soft capsules, or syrups or elixirs. Such compositions may further comprise one or more components such as sweetening agents flavoring agents, coloring agents and preserving agents. Tablets contain the active ingredient in admixture with physiologically acceptable excipients that are suitable for the manufacture of tablets. Such excipients include, for example, inert diluents, granulating and disintegrating agents, binding agents and lubricating agents. Formulations for oral use may also be presented as hard gelatin capsules wherein the active ingredient is mixed with an inert solid diluent, or as soft gelatin capsules wherein the active ingredient is mixed with water or an oil medium. Aqueous suspensions comprise the active materials in admixture with one or more excipients suitable for the manufacture of aqueous suspensions. Such excipients include suspending agents and dispersing or wetting agents. Dispersible powders and granules suitable for preparation of an aqueous suspension by the addition of water provide the active ingredient in admixture with a dispersing or wetting agent, suspending agent and one or more preservatives.

Oily suspensions may be formulated by suspending the active ingredients in a vegetable oil (e.g., arachis oil, olive oil, sesame oil or coconut oil) or in a mineral oil such as liquid paraffin. Pharmaceutical compositions may also be in the form of oil-in-water emulsions. The oily phase may be a vegetable oil or a mineral oil or mixture thereof. Suitable emulsifying agents include, for example, naturally-occurring gums, naturally-occurring phosphatides and anhydrides.

Pharmaceutical compositions may be sterilized by conventional sterilization techniques, or may be sterile filtered. Sterile aqueous solutions may be packaged for use as is, or lyophilized, the lyophilized preparation being combined with a sterile aqueous carrier prior to administration. The pH of an aqueous pharmaceutical composition typically will be between 3 and 11, more preferably between 5 and 9 or between 6 and 8, and most preferably between 7 and 8, such as 7 to 7.5.

Topical compositions according to the present disclosure are in a form suitable for the application to the skin such as e.g. creams, milks, lotions, masks, serums, hydrodispersions, suspensions, cream gels, or gels etc. In some embodiments, the composition comprises at least one physiologically acceptable carrier. In some embodiments, the compositions comprises excipients, diluents, adjuvants, or additives. In some embodiments, the topical composition comprises a thickening agent. The thickening agent can be Arginine, K-carrageenan, Xanthan gum, Laponite, Cetyl alcohol, Stearyl alcohol, Carnauba wax, Stearic acid, Guar gum, Locust bean gum, Gelatin, Silica, Bentonite, Magnesium aluminum silicate, Carbomer 940 or the similar agents or combinations thereof.

Examples of cosmetic or topical excipients, diluents, adjuvants, additives as well as active ingredients commonly used in the skin care industry which are suitable for use in the topical compositions of the present disclosure are for example described in the International Cosmetic Ingredient Dictionary & Handbook by Personal Care Product Council (http://www.personalcarecouncil.org/), accessible by the online INFO BASE (http://online.personalcarecouncil.org/jsp/Home.jsp), without being limited thereto.

In some embodiments the chimeric proteins provided herein are present within a pharmaceutical composition at a concentration such that administration of a single dose to a patient delivers a therapeutically effective amount. A therapeutically effective amount is an amount that results in a discernible patient benefit, such as detectable repair or regeneration of damaged tissue, prevention or diminution of symptoms of tissue damage.

Therapeutically effective amounts can be approximated from the amounts sufficient to achieve detectable tissue repair or regeneration in one or more animal models. Nonetheless, it will be apparent that a variety of factors will affect the therapeutically effective amount, including the activity of the chimeric protein employed; the age, body weight, general health, sex and diet of the patient; the time and route of administration; the rate of excretion; any simultaneous treatment, such as a drug combination; and the type and severity of the tissue damage in the patient undergoing treatment.

Optimal dosages may be established using routine testing, and procedures that are well known in the art. Dosages generally range from about 0.5 mg to about 5,000 mg of chimeric protein per dose (e.g., 0.5 mg, 1 mg, 2 mg, 5 mg, 10 mg, 50 mg, 100 mg, 500 mg, 1,000 mg, 1,500 mg, 2,000 mg, 3,000 mg, 3,500 mg, 4,000 mg, 4,500 mg or 5,000 mg per dose). In general, compositions providing dosage levels ranging from about 0.1 mg to about 100 mg per kilogram of body weight per day are used. For example, about 0.1, 1, 5, 10, 20, 25, 30, 40, 50, 60, 70, 80, 90 or 100 mg per kilogram of body weight per day can be administered. In some embodiments, the method comprises administering between 12 to 25 mg/kg, for example 12, 13, 14, 15, 16, 17, 18, 19, 20, 21, 22, 23, 24 or 25 mg/kg, to the subject in need thereof.

In some embodiments, the therapeutic concentration of the chimeric protein provides a serum/plasma concentration of about 100 ng/mL. In some embodiments, a concentration of 100 ng/mL can effectively drive pro-survival signaling (e.g. phosphorylation of AKT) in damaged tissues.

Pharmaceutical compositions may be packaged for treating or preventing tissue damage. Packaged pharmaceutical preparations include a container holding a therapeutically effective amount of at least one pharmaceutical composition as described herein and instructions (e.g., labeling) indicating that the contained composition is to be used for preventing or treating ARS in a patient. Pharmaceutical compositions may be packaged in multiple single dose units, each containing a fixed amount of chimeric protein in a sealed package. Alternatively, the container may hold multiple doses of the pharmaceutical composition.

### Methods of Treatment

The pharmaceutical compositions can be administered to a patient (e.g. a human) to treat tissue damage in the patient.

As used herein the term “patient” or a “subject” as used interchangeably and refers to a mammal, preferably a human. In some embodiments, the patient is a patient undergoing cancer radiotherapy. In some embodiments, the patient is a patient undergoing diagnostic radiological procedures. In some embodiments, the patient is emergency personnel. In some embodiments, the patient has been diagnosed with ARS.

In some embodiments, the patient is a patient undergoing cancer radiotherapy. Although radiation therapy can be an effective method to control tumor growth, selectively reaching only the cancerous cells/tissue can be a challenge leading to adverse effects, such as toxicity, on healthy tissue.

The term “therapeutically effective amount” refers to the amount or dose of chimeric proteins described herein which, upon single or multiple dose administration to a patient, provides the desired treatment.

Within the context of the present disclosure, the term “treatment” encompasses both radio-protection (i.e. protection from radiation damage), radio-mitigation and therapeutic treatment.

In radio-protecting applications, the pharmaceutical composition as described herein comprising a therapeutically effective amount of the chimeric protein is administered, prior to radiation exposure, to a patient susceptible to or otherwise at risk for developing symptom, disease or condition, in order to prevent, delay or reduce the severity of one or more symptom, disease or condition associated with ionizing radiation. In some embodiments, in radio-protecting applications, the pharmaceutical composition comprising a therapeutically effective amount of the chimeric protein is administered to a patient susceptible to or otherwise at risk for developing pathological tissue/organ damage, in order to prevent, delay or reduce the severity of tissue/organ damage. Radioprotection benefits include but are not limited to reduction of the severity of the symptom(s) associated with ionizing radiation, improved survival of subjects who have been exposed to radiation.

In some embodiments, the ionizing radiation ranges from 2 to 30 Gy. In some embodiments, the ionizing radiation ranges from 2 to 6 Gy. In some embodiments, the ionizing radiation is greater than 1 Gy. In some embodiments, the ionizing radiation is greater than 6 Gy.

In other applications, treatment is performed in order to reduce the severity of one or more symptom, disease or condition associated with ionizing radiation. In therapeutic applications, treatment is performed in order to reduce the severity of the pathological tissue damage or regenerate cells, tissue or organs after damage. Therapeutic benefits include but are not limited to reduction of the severity of the symptom(s) associated with ionizing radiation, improved survival of subjects who have been exposed to radiation, decreased tissue injury, fewer and decreased intensity of GI and hematologic symptoms, less pneumonitis and less fibrosis for lung.

“Treatment”, “treat”, or “treating” as used herein refer to a method of reducing the effects or symptoms of a disease or condition, a method of reducing the disease or condition itself rather than just the symptoms, or a method of reducing tissue/organ damage. The methods of treatment as used herein can refer to a 10%, 20%, 30%, 40%, 50%, 60%, 70%, 80%, 90%, 100% (or any amount therebetween) reduction in the severity of a symptom or disease progression when compared to levels in the same subject before treatment or control subjects. It is understood that “treatment” does not necessarily refer to a cure of the disease or condition, but an improvement in the outlook of a disease or condition.

As used herein, “reduction”, “decrease” or “reduce” refer to any change that results in a smaller amount of a symptom, condition, disease or tissue damage. For example, a reduction or decrease can be a change in the ARS such that the symptoms are less than previously observed. Thus, for example, a reduction or decrease can include but is not limited to a 10%, 20%, 30%, 40%, 50%, 60%, 70%, 80%, 90% or 100% (or any percentage of reduction in between) decrease in the symptoms associated with exposure to ionizing radiation.

In some embodiments, a therapeutically effective amount of the composition comprising the chimeric protein reduces at least one symptom associated with ARS by, e.g., at least 10%, at least 20%, at least 30%, at least 40%, at least 50%, at least 60%, at least 70%, at least 80%, at least 90% or at least 100%. In some embodiments, a therapeutically effective amount of the chimeric protein reduces at least one symptom associated with ARS by, e.g., at least 10%, at least 20%, at least 30%, at least 40%, at least 50%, at least 60%, at least 70%, at least 80%, at least 90% or at least 100%. In some embodiments, a therapeutically effective amount of the chimeric protein disclosed herein reduces at least one symptom associated with ARS by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%. In some embodiments, a therapeutically effective amount of the chimeric protein reduces at least one symptom associated with ARS for, e.g., at least one week, at least one month, at least two months, at least three months, at least four months, at least five months, at least six months, at least seven months, at least eight months, at least nine months, at least ten months, at least eleven months, or at least twelve months.

In some embodiments, the pharmaceutical composition comprising an effective amount of the chimeric protein can be administered in combination with other therapeutic compositions.

Representative pathological tissue damage in ARS includes gastro-intestinal (GI) tissue damage, pulmonary tissue damage, cerebrovascular tissue damage, hemopoietic cells damage, cutaneous tissue damage. In some embodiments, the pharmaceutical composition can be used to protect the tissue/organ from damage and/or to regenerate tissue/organ after tissue or organ damage.

In some embodiments, the pharmaceutical compositions can be administered in combination with existing treatments known in the art.

In some embodiments, the pharmaceutical composition includes, but is not limited to, a composition suitable for oral, rectal, nasal, inhalation, topical (including, but not limited to, dermal, transdermal, buccal and sublingual), vaginal or parenteral (including, but not limited to, subcutaneous, intramuscular, intravenous, intradermal, intraocular and inhalation administration.

In some embodiments, the pharmaceutical composition is suitable for intravenous, intramuscular, subcutaneous, intranasal, intrathecal administration.

In some embodiments, the composition is administered by direct injection. In some embodiments, the composition is formulated for intravenous (IV) injection. In some embodiments, the composition is formulated for IV bolus injection.

### Radio-Protection

Aspects of the disclosure relate to methods for reducing the extent of ionizing radiation induced effects, the method comprising administering a therapeutically effective amount of the chimeric protein prior to ionizing radiation exposure.

In some embodiments, the composition is administered prior to radiation exposure. In some embodiments, the chimeric protein can be administered 7 days, 6 days, 5 days, 4 days, 3 days, 2 days, 1 day, 12-24 hours, 6-12 hours, 3-6 hours etc . . . prior to radiation exposure.

In some embodiments, a therapeutically effective amount generally is in the range of about 0.1 mg/kg to about 100.0 mg/kg per dose. In some embodiments, an effective amount of a protein disclosed herein can be, e.g., about 0.1 mg/kg to about 1 mg/kg per dose, 0.1 mg/kg to about 10 mg/kg per dose, 0.1 mg/kg to about 100 mg/kg per dose, 1 mg/kg to about 100 mg/kg per dose or 10 mg/kg to about 100 mg/kg per dose.

In some embodiments, a therapeutically effective amount generally is in the range of about 0.1 mg/kg to about 1,000.0 mg/kg per day. In some embodiments, an effective amount of a protein disclosed herein can be, e.g., about 0.01 mg/kg to about 0.1 mg/kg per day, 0.01 mg/kg to about 1 mg/kg per day, 0.01 mg/kg to about 10 mg/kg per day, 0.01 mg/kg to about 100.0 mg/kg per day, 0.01 mg/kg to about 200.0 mg/kg per day, 0.1 mg/kg to about 1 mg/kg per day, 0.1 mg/kg to about 10 mg/kg per day, 0.1 mg/kg to about 100 mg/kg per day, 0.1 mg/kg to about 200 mg/kg per day, 1 mg/kg to about 100 mg/kg per day, 1 mg/kg to about 200 mg/kg per day, 10 mg/kg to about 100 mg/kg per day or 10 mg/kg to about 1000 mg/kg per day.

### Radio-Mitigation

Other aspects of the disclosure relate to methods for reducing the extent of ionizing radiation induced effects, the method comprising administering a therapeutically effective amount of the chimeric protein after to radiation exposure. In some embodiments, the administration of the chimeric protein is prior to the onset of symptoms (radio-mitigation).

As used herein, “mitigate” means to reduce the damage associated with a symptom, disease, or condition relative to the untreated state. “Mitigation” can be in reference to a symptom, disease, or condition, in addition to or alternatively to cell, tissue or organ damage associated with the symptom, disease, or condition. Reduction can be a 10, 20, 30, 40, 50, 60, 70, 80, 90 100%, or any amount of reduction in between as compared to untreated, native, or control levels. It is also understood that “mitigation” has occurred if the further damage due to disease progression or symptoms are reduced without a reduction in the state prior to treatment.

In some embodiments, methods for mitigating radiation damage in a subject in need thereof are provided. In some embodiments, after administration of the chimeric protein or pharmaceutical composition provided herein, further damage can be reduced relative to a control that do not undergo the treatment, even if the level of damage in the subject was not reduced relative to pretreatment levels.

In some embodiments, the chimeric protein can be administered at any point after radiation exposure. It should be appreciated that while the animal rule for radio-mitigators generally requires for administration no earlier than 24 hours post exposure, the therapeutic window for mitigation likely opens during the first 24 hours.

In some embodiments, the chimeric protein can be administered between 1 and 24 hours post exposure, between 1 and 24 hours post exposure, between 2 and 24 hours post exposure, between 3 and 24 hours post exposure, between 4 and 24 hours post exposure, between 5 and 24 hours post exposure, between 6 and 24 hours post exposure, between 7 and 24 hours post exposure, between 8 and 24 hours post exposure, between 9 and 24 hours post exposure, between 10 and 24 hours post exposure, between 11 and 24 hours post exposure, between 12 and 24 hours post exposure, between 13 and 24 hours post exposure, between 14 and 24 hours post exposure, between 15 and 24 hours post exposure, between 16 and 24 hours post exposure, between 17 and 24 hours post exposure, between 18 and 24 hours post exposure, between 19 and 24 hours post exposure, between 20 and 24 hours post exposure, between 21 and 24 hours post exposure, between 22 and 24 hours post exposure, between 23 and 24 hours post exposure. For example, the chimeric protein can be administered at about or within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72 hours post exposure.

In some embodiments, a therapeutically effective amount generally is in the range of about 0.1 mg/kg to about 100.0 mg/kg per dose. In some embodiments, an effective amount of a protein disclosed herein can be, e.g., about 0.1 mg/kg to about 1 mg/kg per dose, 0.1 mg/kg to about 10 mg/kg per dose, 0.1 mg/kg to about 100 mg/kg per dose, 1 mg/kg to about 100 mg/kg per dose or 10 mg/kg to about 100 mg/kg per dose.

Dosing can be single dosage or cumulative (serial dosing), and can be readily determined by one skilled in the art. For instance, dosing may comprise a one-time administration of an effective dose of the composition disclosed herein. As a non-limiting example, an effective dose of the composition disclosed herein can be administered once to a patient. Alternatively, dosing may comprise multiple administrations of an effective dose of the chimeric protein or pharmaceutical composition disclosed herein carried out over a range of time periods, such as, e.g., from one time daily, two times daily, three times daily, four times daily, five times daily, six times daily, seven times daily, eight times daily, nine times daily, ten times daily, eleven times daily, to twelve times daily, or more.

In some embodiments, one or more single doses of chimeric protein or the pharmaceutical composition can be administered to the subject within 24 hours following the exposure of the subject to the radiation. In some embodiments, one or more single doses of chimeric protein or the pharmaceutical composition can be administered beginning within 24 hours following the exposure of the subject to the radiation followed by a daily regimen.

In some embodiments, an effective dose of the chimeric protein or the pharmaceutical composition can be administered one to 10 times (e.g. 1, 2, 3, 4, 5, 6, 7, 8, 9, or 10 times) daily following the exposure of the subject to the ionizing radiation and for up to 100 days or more. some embodiments, an effective dose of the chimeric protein or the pharmaceutical composition can be administered one to 10 times (e.g. 1, 2, 3, 4, 5, 6, 7, 8, 9, or 10 times) daily following the exposure of the subject to the ionizing radiation and for up until the subject no longer is need of therapy (e.g. complete blood counts, bone marrow aspiration and biopsy, lung tests (x-ray, CT scan) and biopsy, GI test (endoscopy, biopsy) are normal).

In some embodiments, a therapeutically effective amount generally is in the range of about 0.1 mg/kg to about 1,000.0 mg/kg per day. In some embodiments, an effective amount of a protein disclosed herein can be, e.g., about 0.01 mg/kg to about 0.1 mg/kg per day, 0.01 mg/kg to about 1 mg/kg per day, 0.01 mg/kg to about 10 mg/kg per day, 0.01 mg/kg to about 100.0 mg/kg per day, 0.01 mg/kg to about 200.0 mg/kg per day, 0.1 mg/kg to about 1 mg/kg per day, 0.1 mg/kg to about 10 mg/kg per day, 0.1 mg/kg to about 100 mg/kg per day, 0.1 mg/kg to about 200 mg/kg per day, 1 mg/kg to about 100 mg/kg per day, 1 mg/kg to about 200 mg/kg per day, 10 mg/kg to about 100 mg/kg per day or 10 mg/kg to about 1000 mg/kg per day.

### Treatment of ARS

In some embodiments, methods for treating ARS in a subject in need thereof are provided. In some embodiments, the method comprises administering the chimeric protein is after the onset of symptoms.

It should be appreciated that the timing of administration can depend upon such factors as the severity of the subject's symptoms. For example, an effective dose of the composition disclosed herein can be administered to a patient once daily, multiple times (e.g. 1, 2, 3, 4, 5, 6, 7, 8, 9, 10, 11 or 12 times) a day, once every other day, once every 2 days, once every 3 days, once every four days, once every five days, once every six days, once a week, once a month for an indefinite period of time, or until the subject no longer requires therapy (e.g. complete blood counts, bone marrow aspiration and biopsy, lung tests (x-ray, CT scan) and biopsy, GI test (endoscopy, biopsy) are normal).

In some embodiments, the chimeric protein can be administered 1 day or more post exposure. For example, the chimeric protein can be administered at about or within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72 days or more post exposure.

In some embodiments, a therapeutically effective amount generally is in the range of about 0.1 mg/kg to about 100.0 mg/kg per dose. In some embodiments, an effective amount of a protein disclosed herein can be, e.g., about 0.1 mg/kg to about 1 mg/kg per dose, 0.1 mg/kg to about 10 mg/kg per dose, 0.1 mg/kg to about 100 mg/kg per dose, 1 mg/kg to about 100 mg/kg per dose or 10 mg/kg to about 100 mg/kg per dose.

Dosing can be single dosage or cumulative (serial dosing), and can be readily determined by one skilled in the art. For instance, treatment of ARS may comprise a one-time administration of an effective dose of the composition disclosed herein. As a non-limiting example, an effective dose of the composition disclosed herein can be administered once to a patient. Alternatively, treatment of ARS may comprise multiple administrations of an effective dose of the chimeric protein or pharmaceutical composition disclosed herein carried out over a range of time periods, such as, e.g., from one time daily to ten times daily. For example, a single dose of the chimeric protein or the pharmaceutical composition can be administered one to 10 times (e.g. 1, 2, 3, 4, 5, 6, 7, 8, 9, or 10 times) daily following the exposure of the subject to the ionizing radiation. In some embodiments, administration of an effective dose of chimeric protein or the pharmaceutical composition can be once every other day, once every few days, weekly, monthly or yearly.

In some embodiments, an effective dose of the chimeric protein or the pharmaceutical composition can be administered one to 10 times (e.g. 1, 2, 3, 4, 5, 6, 7, 8, 9, or 10 times) daily following the exposure of the subject to the ionizing radiation and for up to 100 days or more. In some embodiments, an effective dose of the chimeric protein or the pharmaceutical composition can be administered one to 10 times (e.g. 1, 2, 3, 4, 5, 6, 7, 8, 9, or 10 times) daily following the exposure of the subject to the ionizing radiation and until the subject no longer requires therapy (e.g. complete blood counts, bone marrow aspiration and biopsy, lung tests (x-ray, CT scan) and biopsy, GI test (endoscopy, biopsy) are normal).

In some embodiments, a therapeutically effective amount generally is in the range of about 0.1 mg/kg to about 1,000.0 mg/kg per day. In some embodiments, an effective amount of a protein disclosed herein can be, e.g., about 0.01 mg/kg to about 0.1 mg/kg per day, 0.01 mg/kg to about 1 mg/kg per day, 0.01 mg/kg to about 10 mg/kg per day, 0.01 mg/kg to about 100.0 mg/kg per day, 0.01 mg/kg to about 200.0 mg/kg per day, 0.1 mg/kg to about 1 mg/kg per day, 0.1 mg/kg to about 10 mg/kg per day, 0.1 mg/kg to about 100 mg/kg per day, 0.1 mg/kg to about 200 mg/kg per day, 1 mg/kg to about 100 mg/kg per day, 1 mg/kg to about 200 mg/kg per day, 10 mg/kg to about 100 mg/kg per day or 10 mg/kg to about 1000 mg/kg per day. In some embodiments, the pharmacological compositions described herein can further include one or more additional bioactive agents or components to aid in the treatment of damaged tissue or cells and/or facilitate the tissue regenerative process.

Aspects of the disclosure relate to the method comprising administering a chimeric protein comprising targeting domain comprising a variant of human Annexin 5 (AnxV), an activator domain comprising a variant of human insulin-like growth factor IGF-1 and optionally half-life modulator peptide. In some embodiments, the variant of human Annexin 5 (AnxV) comprises one or more mutations, wherein the one or more mutations consist of a substitution at the position corresponding to C316 and optionally at one or more positions corresponding to R63, K70, K101, E138, D139, N160, and combinations thereof. In some embodiments, the variant of human insulin-like growth factor IGF-1 comprises one or more mutations, wherein the one or more mutations consist of a substitution at one or more positions corresponding to E3, Y24, Y31, Y60, and combinations thereof. In some embodiments, the variant of IGF-1 decreases activation of the IGF-1 receptor relative to the wild-type IGF-1. In some embodiments, the half-life modulator peptide comprises a variant of human serum albumin (HSA) comprising one or more mutations, wherein the one or more mutations consist of a substitution at one or more positions corresponding to C58 and N527, and combinations thereof.

In some embodiments, administration of the chimeric proteins result in one or more of the following: modulation of cell proliferation, cell survival, tissue regeneration, decreased fibrosis, decreased inflammation.

Provided herein is a composition for use in a method of effecting radioprotection, comprising administering the composition comprising an effective amount of a chimeric protein to a subject in need thereof, wherein the chimeric protein comprises a targeting domain comprising human annexin 5 or variant thereof, and an activator domain comprising insulin-like growth factor (IGF-1), neuregulin (NRG) or variant thereof, wherein the composition is administered to the subject between 3 hours to 7 days prior to radiation exposure. In some embodiments, the administration of the chimeric protein effects radioprotection in the subject in need thereof.

Provided herein is a composition for use in a method of mitigating radiation injury, comprising administering the composition comprising an effective amount of a chimeric protein to a subject in need thereof, wherein the chimeric protein comprises a targeting domain comprising human annexin 5 (AnxV) or variant thereof, and an activator domain comprising insulin-like growth factor (IGF-1), neuregulin (NRG) or variant thereof, wherein the composition is administered once or multiple times a day to the subject between 1 hour and 21 days after radiation exposure. In some embodiments, the administration of the chimeric protein mitigates radiation injury in the subject in need thereof.

Provided herein is a composition for use in a method of effecting radioprotection and mitigating injury from radiation, comprising administering the composition comprising an effective amount of a chimeric protein to a subject in need thereof, wherein the chimeric protein comprises a targeting domain comprising human annexin 5 (AnxV) or variant thereof, and an activator domain comprising insulin-like growth factor (IGF-1), neuregulin (NRG) or variant thereof, wherein the composition is initially administered to the subject between 3 hours to 7 days prior to the radiation exposure, wherein the composition is subsequently administered once a day or multiple times a day to the subject between 1 hour and 21 days after radiation exposure. In some embodiments, the administration of the chimeric protein effects radioprotection and mitigates injury from radiation in the subject in need thereof.

Provided herein is a composition in a method for treating acute radiation syndrome (ARS), comprising administering the composition comprising a therapeutically effective amount of a chimeric protein to a subject in need thereof, wherein the chimeric protein comprises a targeting domain comprising human annexin 5 (AnxV) or variant thereof, and an activator domain comprising insulin-like growth factor (IGF-1), neuregulin (NRG) or variant thereof, wherein the composition is administered to the subject within 1 to 72 days after radiation exposure. In some embodiments, the administration of the chimeric proteins results in the amelioration of one or more symptoms of ARS.

Provided herein is a chimeric protein for use in a method of effecting radioprotection, comprising administering an effective amount of the chimeric protein to a subject in need thereof, wherein the chimeric protein comprises a targeting domain comprising human annexin 5 or variant thereof, and an activator domain comprising insulin-like growth factor (IGF-1), neuregulin (NRG) or variant thereof, wherein the chimeric protein is administered to the subject between 3 hours to 7 days prior to radiation exposure. In some embodiments, the administration of the chimeric protein effects radioprotection in the subject in need thereof.

Provided herein is a chimeric protein for use in a method of mitigating radiation injury, comprising administering an effective amount of the chimeric protein to a subject in need thereof, wherein the chimeric protein comprises a targeting domain comprising human annexin 5 (AnxV) or variant thereof, and an activator domain comprising insulin-like growth factor (IGF-1), neuregulin (NRG) or variant thereof, wherein the chimeric protein is administered once or multiple times a day to the subject between 1 hour and 21 days after radiation exposure. In some embodiments, the administration of the chimeric protein mitigates radiation injury in the subject in need thereof.

Provided herein is a chimeric protein for use in a method of effecting radioprotection and mitigating injury from radiation, comprising administering an effective amount of the chimeric protein to a subject in need thereof, wherein the chimeric protein comprises a targeting domain comprising human annexin 5 (AnxV) or variant thereof, and an activator domain comprising insulin-like growth factor (IGF-1), neuregulin (NRG) or variant thereof, wherein the chimeric protein is initially administered to the subject between 3 hours to 7 days prior to the radiation exposure, wherein the composition is subsequently administered once a day or multiple times a day to the subject between 1 hour and 21 days after radiation exposure. In some embodiments, the administration of the chimeric protein effects radioprotection and mitigates injury from radiation in the subject in need thereof.

Provided herein is a chimeric protein in a method for treating acute radiation syndrome (ARS), comprising administering a therapeutically effective amount of the chimeric protein to a subject in need thereof, wherein the chimeric protein comprises a targeting domain comprising human annexin 5 (AnxV) or variant thereof, and an activator domain comprising insulin-like growth factor (IGF-1), neuregulin (NRG) or variant thereof, wherein the chimeric protein is administered to the subject within 1 to 72 days after radiation exposure. In some embodiments, the administration of the chimeric proteins results in the amelioration of one or more symptoms of ARS.

In some embodiments, the variant of human Annexin 5 (AnxV) comprises one or more mutations, wherein the one or more mutations consist of a substitution at the position corresponding to C316 and optionally at one or more positions corresponding to R63, K70, K101, E138, D139, N160, and combinations thereof. In some embodiments, the variant of human insulin-like growth factor IGF-1 comprises one or more mutations, wherein the one or more mutations consist of a substitution at one or more positions corresponding to E3, Y24, Y31, Y60, and combinations thereof. In some embodiments, the variant of IGF-1 decreases activation of the IGF-1 receptor relative to the wild-type IGF-1. In some embodiments, the half-life modulator peptide comprises a variant of human serum albumin (HSA) comprising one or more mutations, wherein the one or more mutations consist of a substitution at one or more positions corresponding to C58 and N527, and combinations thereof.

### EXAMPLES

The following Examples are offered by way of illustration and not by way of limitation. Unless otherwise specified, all reagents and solvents are of standard commercial grade and are used without further purification. Using routine modifications, the procedures provided in the following Examples may be varied by those of ordinary skill in the art to make and use other chimeric proteins and pharmaceutical compositions within the scope of the present disclosure.

### Example 1. Scp776—Mediated Radioprotection

In the event of a nuclear disaster, emergency response personnel may be required to enter an area where radioactive contamination from nuclear fallout is dangerous. Prior to entering this zone, emergency response personnel are dosed with chimeric protein scp776. As radiation exposure leads to apoptosis, phosphatidylserine is exposed on the cell surfaces of damaged tissues. With a prophylactic level of scp776 in circulation, cells entering apoptosis are targeted by scp776, leading to specific activation of the receptor tyrosine kinase IGF1-R. The transmission of the pro-survival signals promotes exit from apoptosis, thereby limiting damage from radiation exposure and increasing the time that emergency response personnel can safely operate in the nuclear fallout zone.

Subjects pre-treated with scp776 demonstrate less severe cytopenias, improved gut function, less lung pneumonitis, less lung fibrosis, improved renal function or any combination of the foregoing as compared with subjects not pre-treated with scp776.

### Example 2. Scp776—Mediated Radiomitigation

In the event of a nuclear disaster, persons located within the nuclear fallout zone are subjected to harmful radiation. Following evacuation from the nuclear fallout zone, exposed persons are dosed with therapeutic levels of scp776. Because the primary mode of tissue damage resulting from radiation exposure is apoptosis, a number of tissues are targeted by scp776. Targeted delivery of pro-survival signals to those and the surrounding tissues promotes exit from apoptosis.

Subjects treated with scp776 after radiation exposures demonstrate less severe cytopenias, improved gut function, less lung pneumonitis, less lung fibrosis, improved renal function or any combination of the foregoing as compared with subjects not treated with scp776.

### Example 3. Scp776—Treatment of ARS

Subjects diagnosed with ARS are dosed with therapeutic levels of scp776. After a suitable period of time, the subjects demonstrate less severe cytopenias, improved gut function, less lung pneumonitis, less lung fibrosis, improved renal function or any combination of the foregoing as compared with ARS subjects not treated with scp776.

### Example 4. Scp776 Protection and Mitigation of Gastrointestinal Acute Radiation Syndrome

A study of the survival distributions of two groups of mice that were subjected to a whole body irradiation dose, with partial shielding, of 1650 cGy, at a targeted dose rate of 53 cGy per minute was performed.

The group of control mice were subjected to a whole body irradiation dose, with partial shielding, of 1650 cGy, at a targeted dose rate of 53 cGy per minute. The mean exposures for the three nanodot radiation counters were 1658, 1616, and 160 cGy while the mean Farmer ionization chamber reading was 1669 cGy.

The group of scp776 treated mice were subjected to a whole body irradiation dose, with partial shielding, of 1650 cGy, at a targeted dose rate of 53 cGy per minute. The mean exposures for the three nanodot radiation counters were 1657, 1613, and 160 cGy while the mean Farmer ionization chamber reading was 1669 cGy.

FIG. 2 shows the log-rank test comparing the survival distributions of two groups of mice that were subjected to a whole body irradiation dose, with partial shielding, of 1650 cGy, at a targeted dose rate of 53 cGy per minute. The primary endpoint for the blinded study was survival. The group of mice labeled as Placebo received twice daily intraperitoneal injections of formulation buffer, while the group of mice labeled as Scp776 received twice daily intraperitoneal injections of formulation buffer containing Scp776. Administrations of placebo and scp776 were provided twice daily beginning the day prior to radiation exposure and continuing through day 6 post radiation exposure. Of the 28 mice that received Placebo, ten perished by day 8. For the 28 mice that received Scp776, only four perished by day 8. The therapeutic potential of the test item, scp776, for the protection and mitigation of gastrointestinal acute radiation syndrome (GI-ARS) is demonstrated therein.

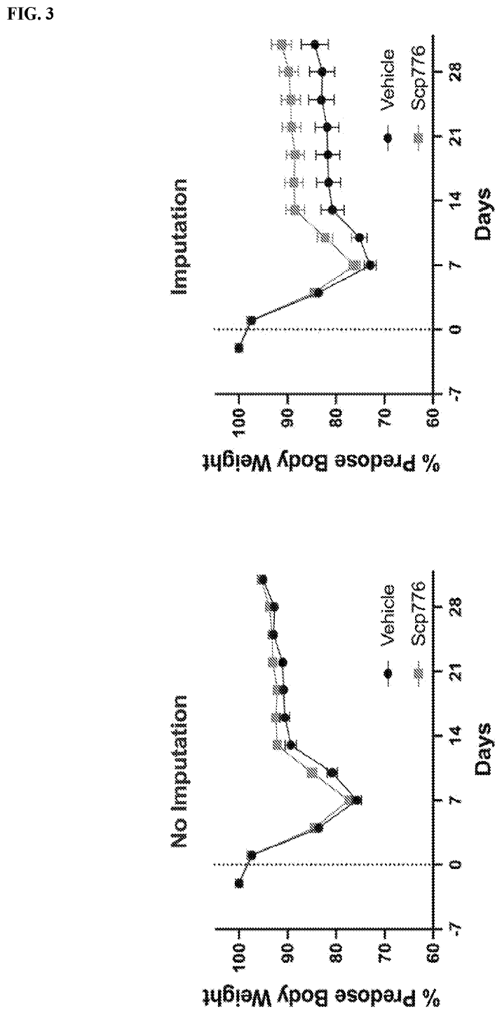

FIG. 3 shows the change in body weight for the two groups of mice that were subjected to a whole body irradiation dose, with partial shielding, of 1650 cGy, at a targeted dose rate of 53 cGy per minute. A secondary endpoint for the blinded study was maintenance of body weight. Plotted individual body weight measurements were normalized to each animal's pre-dose weight and the mean±standard error of the mean are represented by the boxes and error bars, respectively. The left panel shows the comparison of body weights for the two groups without imputation, where the body weights following death are censored from the analysis. The right panel shows the comparison of body weights for the two groups with imputation, where the body weights following death are imputed to the lowest observed body weight in any animal. Statistical analyses comparing the time courses of body weight change showed a significant improvement in maintenance of body weight in the animals that received scp776 (p=0.028 via Mixed Effects MLM without imputation; p=0.024 via Repeated Measures ANOVA with imputation).

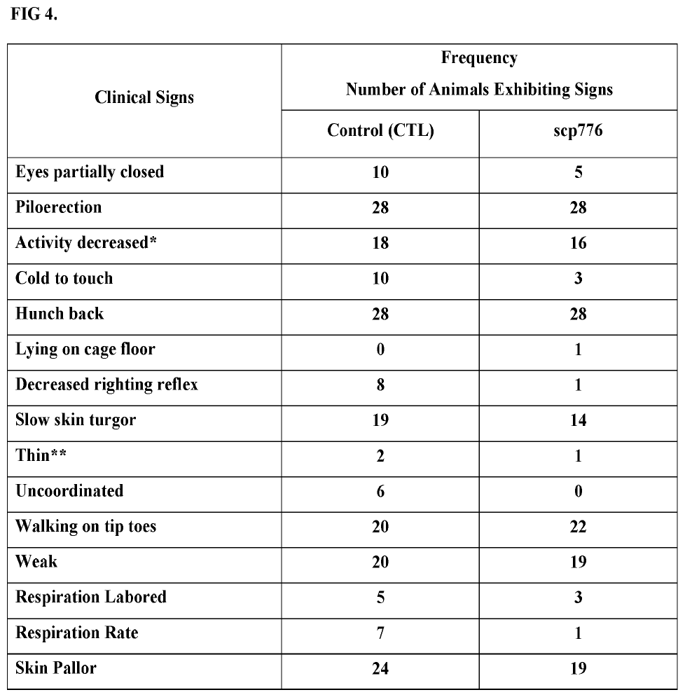

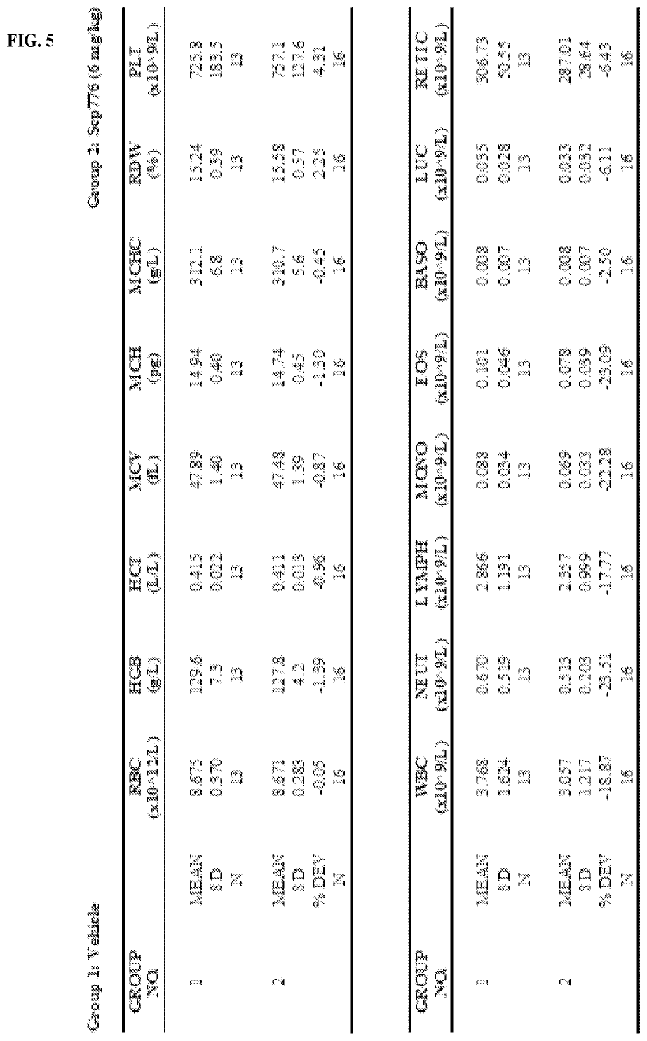

FIG. 4 shows the frequency of clinical signs observed for the two groups of mice that were subjected to a whole body irradiation dose, with partial shielding, of 1650 cGy, at a targeted dose rate of 53 cGy per minute. The clinical signs were recorded daily in a blinded manner and the frequency is tabulated here. Of note, the duration of “Activity decreased” in the control group was nine days and the duration of the “Activity decreased” in the group that received scp776 was only three days. Furthermore, the observation of “Thin” had durations of 6 days and 1 day for the control and scp776 groups, respectively.

FIG. 5 shows the clinical laboratory results for the two groups of mice that were subjected to a whole body irradiation dose, with partial shielding, of 1650 cGy, at a targeted dose rate of 53 cGy per minute. While there is a slight trend in decreased white blood cell counts for the scp776 group relative to the control group, overall the laboratory values are comparable.

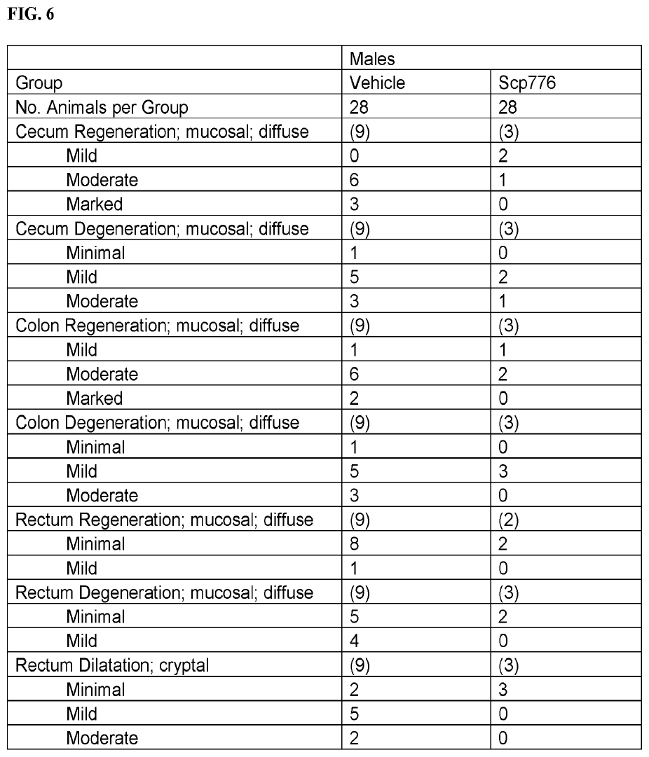

FIG. 6 tabulates the macroscopic observations of intestinal damage for the two groups of mice that were subjected to a whole body irradiation dose, with partial shielding, of 1650 cGy, at a targeted dose rate of 53 cGy per minute. Following the irradiation dose, moribund mice were euthanized (Vehicle, n=9; Scp776, n=3) and the cecum, colon, and rectum of animals were examined for macroscopic evidence and degree of regeneration (healing of injury), degeneration (worsening of injury), and dilatation (widening of the intestinal tract). The findings in FIG. 6 are deemed abnormal as the examination of an uninjured animal would not register findings in this analysis. In comparison of the Vehicle and Scp776 treated groups, there is evidence of a marginal trend towards higher severity grades for abnormal findings in control animals (e.g., Rectum Degeneration, Vehicle: 0% Normal, 55% Minimal, 45% Mild vs. Scp776: 33% Normal, 66% Minimal, 0% Mild; Rectum Dilatation, Vehicle: 0% Normal, 22% Minimal, 55% Mild, 22% Moderate vs. Scp776: 0% Normal, 100% Minimal, 0% Mild, 0% Moderate). These results could indicate that the large intestine has an earlier response to the effect of scp776 and/or less damage.

### INCORPORATION BY REFERENCE

All publications, patents and sequence database entries mentioned herein are hereby incorporated by reference in their entirety as if each individual publication or patent was specifically and individually indicated to be incorporated by reference.

What is claimed is:

1. A method of effecting radioprotection, the method comprising:
administering a composition comprising an effective amount of a chimeric protein to provide radioprotection to a subject in need thereof,
wherein the chimeric protein comprises a targeting domain comprising human annexin 5 or variant thereof, and an activator domain comprising insulin-like growth factor (IGF-1), neuregulin (NRG) or variant thereof, and
wherein the composition is administered to the subject between 3 hours to 7 days prior to radiation exposure.

2. A method of mitigating radiation injury, the method comprising:
administering a composition comprising an effective amount of a chimeric protein to a subject in need thereof,
wherein the chimeric protein comprises a targeting domain comprising human annexin 5 (AnxV) or variant thereof, and an activator domain comprising insulin-like growth factor (IGF-1), neuregulin (NRG) or variant thereof, and
wherein the composition is administered once or multiple times a day to the subject between 1 hour and 21 days after radiation exposure.

3. A method of effecting radioprotection and mitigating injury from radiation, the method comprising:
administering a composition comprising an effective amount of a chimeric protein to a subject in need thereof,
wherein the chimeric protein comprises a targeting domain comprising human annexin 5 (AnxV) or variant thereof, and an activator domain comprising insulin-like growth factor (IGF-1), neuregulin (NRG) or variant thereof,
wherein the composition is initially administered to the subject between 3 hours to 7 days prior to radiation exposure, and
wherein the composition is subsequently administered once a day or multiple times a day to the subject between 1 hour and 21 days after the radiation exposure.

4. A method of treating acute radiation syndrome (ARS), the method comprising:
administering a composition comprising a therapeutically effective amount of a chimeric protein to a subject in need thereof,
wherein the chimeric protein comprises a targeting domain comprising human annexin 5 (AnxV) or variant thereof, and an activator domain comprising insulin-like growth factor (IGF-1), neuregulin (NRG) or variant thereof, and
wherein the composition is administered to the subject within 1 to 72 days after radiation exposure.

5. The method of claim 1, claim 2, claim 3, or claim 4, wherein the chimeric protein further comprising a peptide, wherein the peptide extends the half-life of the chimeric protein.

6. The method of claim 1, claim 2, claim 3 or claim 4, wherein the chimeric protein is targeted to a cell exposing phosphatidyl serine at its surface.

7. The method of claim 1, claim 2, claim 3 or claim 4, wherein the chimeric protein is targeted to an apoptotic cell of an organ of the subject.

8. The method of claim 1, claim 2, claim 3 or claim 4, wherein the targeting domain is a non-internalizing variant of annexin 5.

9. The method of claim 8, wherein the chimeric protein is substantially not internalized by cells.

10. The method of claim 8, wherein the non-internalizing variant of annexin 5 comprises one or more mutations, wherein the one or more mutations comprises a substitution at a position corresponding to C316 and optionally at one or more positions corresponding to R63, K70, K101, E138, D139, N160, and combinations thereof.

11. The method of claim 1, claim 2, claim 3 or claim 4, wherein the activator domain of chimeric protein is a variant of human insulin-like growth factor IGF-1 comprising one or more mutations, wherein the one or more mutations consist of a substitution at one or more positions corresponding to E3, Y24, Y31, Y60, and combinations thereof.

12. The method of claim 1, claim 2, claim 3 or claim 4, wherein the chimeric protein further comprises a half-life modulator comprising a variant of human serum albumin (HSA) comprising one or more mutations, wherein the one or more mutations consist of a substitution at one or more positions corresponding to C58 and N527, and combinations thereof.

13. The method of claim 1, claim 2, claim 3 or claim 4, wherein the effective amount is between about 0.1 mg/kg to about 100 mg/kg.

14. The method of claim 1, claim 2, claim 3 or claim 4, wherein the effective amount is between about 12 mg/kg to 25 mg/kg.

15. The method of claim 1, claim 2, claim 3 or claim 4, wherein the effective amount is effective to mitigate or treat radiation injury or ARS in the subject.

16. The method of claim 1, claim 2, claim 3 or claim 4, wherein administration of the composition increases the subject's chance of survival following exposure to radiation.

17. The method of claim 1, claim 2, claim 3 or claim 4, wherein the subject in need thereof received a dose of radiation that is lethal to the subject in absence of administration of the composition.

18. The method of claim 1, claim 2, claim 3 or claim 4, wherein the radiation exposure comprises ionizing radiation exposure.

19. The method of claim 4 or claim 15, wherein ARS is one or more of gastrointestinal (GI-ARS), hematopoietic (H-ARS), pulmonary (P-ARS), cutaneous (C-ARS), and cerebrovascular (B-ARS).

20. The method of claim 1, claim 2, claim 3 or claim 4, wherein the subject is a human.

21. The method of claim 1, claim 2, claim 3 or claim 4, wherein the composition further comprises at least one physiologically acceptable carrier.

22. The method of claim 1, claim 2, claim 3 or claim 4, wherein the chimeric protein is IGF1(E3R/Y31A)_lk7_HSA26-609(C58S/N527Q)_lk7_AnxV2-320(R63A/K70A/K101A/E138A/D139G/N160A/C316A).

23. A composition for use in a method of effecting radioprotection, comprising:
administering the composition comprising an effective amount of a chimeric protein to a subject in need thereof,
wherein the chimeric protein comprises a targeting domain comprising human annexin 5 or variant thereof, and an activator domain comprising insulin-like growth factor (IGF-1), neuregulin (NRG) or variant thereof, and
wherein the composition is administered to the subject between 3 hours to 7 days prior to radiation exposure.

24. A composition for use in a method of mitigating radiation injury, comprising:
administering the composition comprising an effective amount of a chimeric protein to a subject in need thereof,
wherein the chimeric protein comprises a targeting domain comprising human annexin 5 (AnxV) or variant thereof, and an activator domain comprising insulin-like growth factor (IGF-1), neuregulin (NRG) or variant thereof, and
wherein the composition is administered once or multiple times a day to the subject between 1 hour and 21 days after radiation exposure.

25. A composition for use in a method of effecting radioprotection and mitigating injury from radiation, comprising:
administering the composition comprising an effective amount of a chimeric protein to a subject in need thereof,
wherein the chimeric protein comprises a targeting domain comprising human annexin 5 (AnxV) or variant thereof, and an activator domain comprising insulin-like growth factor (IGF-1), neuregulin (NRG) or variant thereof,
wherein the composition is initially administered to the subject between 3 hours to 7 days prior to radiation exposure, and
wherein the composition is subsequently administered once a day or multiple times a day to the subject between 1 hour and 21 days after the radiation exposure.

26. A composition in a method for treating acute radiation syndrome (ARS), comprising:
administering the composition comprising a therapeutically effective amount of a chimeric protein to a subject in need thereof,
wherein the chimeric protein comprises a targeting domain comprising human annexin 5 (AnxV) or variant thereof, and an activator domain comprising insulin-like growth factor (IGF-1), neuregulin (NRG) or variant thereof,
wherein the composition is administered to the subject within 1 to 72 days after radiation exposure.
